(19) United States
(12) Patent Application Publication
(10) Pub. No.: US 2013/0330367 A1
(43) Pub. Date: Dec. 12, 2013

(54) COMPOSITIONS WITH ENHANCED IMMUNOGENICITY AND/OR REDUCED REACTOGENICITY

(71) Applicant: Vaxinnate Corporation, Cranbury, NJ (US)

(72) Inventors: Langzhou Song, Cranbury, NJ (US); Ge Liu, Cranbury, NJ (US); Scott Umlauf, Cranbury, NJ (US); Uma Kavita, Cranbury, NJ (US); Hong Li, Cranbury, NJ (US); Xiangyu Liu, Cranbury, NJ (US); Bruce Weaver, Cranbury, NJ (US); Lynda Tussey, Cranbury, NJ (US)

(73) Assignee: Vaxinnate Corporation, Cranbury, NJ (US)

(21) Appl. No.: 13/970,918

(22) Filed: Aug. 20, 2013

Related U.S. Application Data
(63) Continuation of application No. PCT/US2012/000099, filed on Feb. 21, 2012.
(60) Provisional application No. 61/444,805, filed on Feb. 21, 2011.
(60) Provisional application No. 61/468,894, filed on Mar. 29, 2011.

(51) Int. Cl. C07K 14/195; C07K 14/005

(52) U.S. Cl. CPC C07K 14/195; C07K 14/005
USPC 424/185.1; 530/350

(57) ABSTRACT

The present invention relates to improved vaccines and the design and making of such vaccines that enhance immunogenicity of the vaccine and/or reduce reactogenicity to the vaccine when administered. In particular the vaccines and immunogenic compositions of the present invention relate to flagellin-antigen fusion proteins in which the spatial orientation of the flagellin to antigen and the charge distribution of the antigen is optimized to enhance immunogenicity and/or reduce reactogenicity. The present invention also relates to methods of evaluating the vaccines by measuring the relative expression of certain gene markers. Altered expression of the genes relative to flagellin control sample may indicate that the vaccine is suitable to stimulate an adaptive immune response to the antigen component in the subject with minimal side effects.

### RELATED APPLICATIONS

This application is a continuation of International Application No. PCT/US2012/000099, which designated the United States and was filed on Feb. 21, 2012, published in English, which claims priority to U.S. Provisional Application No. 61/444,805, filed Feb. 21, 2011 and U.S. Provisional Application No. 61/468,894, filed Mar. 29, 2011. The entire teachings of the above applications are incorporated herein by reference.

### INCORPORATION BY REFERENCE OF MATERIAL IN ASCII TEXT FILE

This application incorporates by reference the Sequence Listing contained in the following ASCII text file being submitted concurrently herewith:
a) File name: 37101052002SEQUENCELISTING.txt; created Aug. 7, 2013, 191 KB in size.

### FIELD OF THE INVENTION

The present invention relates to improved vaccines and the design and making of such vaccines that enhance immunogenicity of the vaccine and/or reduce potential reactogenicity to the vaccine when administered. In particular the vaccines and immunogenic compositions of the present invention relate to flagellin-antigen fusion proteins in which the spatial orientation of the flagellin to antigen and the charge distribution of the antigen are optimized to enhance immunogenicity and/or reduce reactogenicity. The invention further relates to methods of assessing suitability of a composition that includes at least one flagellin component and at least one antigen component for use to stimulate an adaptive immune response to the antigen component in a subject with minimal side effects, comprising the step of measuring expression of genetic markers.

### BACKGROUND OF THE INVENTION

A number of influenza vaccine formats which fuse subunits of the hemagglutinin (HA) protein to flagellin have been developed. HA is the major protective antigen for influenza and a subunit of HA referred to as HA1-2 appears to be the minimally protective subunit as demonstrated in preclinical lethal challenge models. A longer subunit referred to as HA1-1(see FIG. 1) genetically fused to flagellin has also been shown to be protective in the preclinical models. For certain subtypes of influenza, a genetic fusion of an even longer head domain, referred to as HA1s, to flagellin is more immunogenic than the shorter subunits.

In addition to using different lengths of the HA antigen, vaccine formats which differ in the attachment point of the vaccine antigen to flagellin have also been developed (see FIG. 2). Some formats carry two copies of the antigen. C-terminal format type vaccines genetically fuse the vaccine antigen to the C terminus of flagellin. R3 format vaccines replace domain 3 by genetically fusing the vaccine antigen to D2, R3.2x format vaccines fuse one copy of the vaccine antigen to the C terminus while an additional copy of the antigen replaces domain 3. Each of these different vaccine formats has different properties. More specifically, the attachment point, or location, of the antigen relative to flagellin can influence the antigenic, the immunogenic and even the reactogenic properties of the vaccine.

The different vaccine formats are thought to influence immunogenicity and reactogenicity by modulating the TLR5 agonist properties of flagellin and/or enhancing the display of the vaccine antigen to immune cells. H1 and H5 influenza subtype vaccines of the R3 and R3.2x formats are highly immunogenic, protective in preclinical challenge models and are also tolerated to higher doses than the equally immunogenic but more reactogenic C-terminal formats. For the influenza B and H3 subtypes however, the R3 and R3.2x vaccine formats are poorly immunogenic.

Therefore, there is a need to produce vaccines coupled to flagellin that are more immunogenic and less reactogenic particularly for influenza subtypes other than H1 and H5. There is also a need to develop methods for screening immunogenic compositions for immunogenicity and reactogenicity that does not require the cost, resources and time required by current animal models.

### SUMMARY OF THE INVENTION

The present invention relates to immunological compositions comprising flagellin and at least one antigen in which the length of the antigen, the charge and/or hydrophobicity of the antigen and the orientation of the antigen to the flagellin is altered such that the compositions are more immunogenic and/or less reactogenic.

The present invention describes new vaccines and immunologic compositions in which the relative orientation of the antigen to the flagellin is altered such that the vaccine is more immunogenic and/or less reactogenic.

The present invention describes new vaccine formats including R23 which replaces domains 2 and 3 of flagellin with the HA head domain thereby altering the spatial relationship of the head domain.

The present invention describes an immunologic fusion protein comprising flagellin and HA linked together by a linker wherein the length of the linker and charge of the linker is optimized to increase immunogenicity and/or reduce reactogenicity of the fusion protein.

The present invention describes a method of improving the antigenicity and immunogenicity of flagellin-antigen fusion proteins comprising optimizing the spatial orientation of the antigen to the flagellin by changing the linker length and/or charge such that a TLR5 binding site on the flagellin is not blocked and the antigen is freely presented.

The present invention describes a method of improving the antigenicity and immunogenicity of flagellin-antigen fusion proteins comprising optimizing the charge distribution of the antigen such that a TLR5 binding site on the flagellin is not blocked and the antigen is freely presented.

The present invention describes a method of improving the antigenicity and immunogenicity of flagellin-antigen fusion proteins comprising decreasing the pI of the antigen such that a TLR5 binding site on the flagellin is not blocked and the antigen is freely presented.

The present invention describes an immunologic composition comprising a flagellin and an antigen wherein the pI of the antigen has been altered such that the pI of the altered antigen is less than the pI of the unaltered antigen.

The present invention also relates to methods of screening compositions that include a portion of flagellin that have minimal side effects such as reactogenicity, and have efficient immunogenicity to the antigen component of the composition.

The invention also relates to methods of assessing suitability of a composition that includes at least one flagellin component and at least one antigen component for use to stimulate an adaptive immune response to the antigen component in a subject with minimal side effects, comprising the step of measuring expression of genes & serum cytokine levels selected from the group consisting of Ccl2, Cd80, Csf2, Cxcl10, Fadd, Hspa1a, IL-1a, IL-1b, IL-6, Irf3, Mapk8ip3, Nf-kb2, Nf-rkb, Ppara, Eif2ak2, Tbk1, T1r2, T1r3, T1r4, T1r7, Tnf, Tnfaip3, Traf6, Jun, Cebpb, Fos, Ptgs and Ticam and IL6 & TNF respectively in a test sample wherein a defined minimum & maximum expression of the genes and cytokines in the test sample relative to a flagellin control sample indicates that the composition is suitable for use to stimulate the adaptive immune response to the antigen component in the subject with minimal side effects.

The invention relates to methods of assessing the suitability of a composition that includes at least one flagellin component and at least one antigen component for use to stimulate an adaptive immune response to the antigen component in a subject with minimal side effects, comprising the step of measuring expression of genes selected from the group listed in FIG. 8 in a test sample wherein altered expression such as underexpression of the genes or cytokines in the test sample relative to a flagellin control sample indicates that the composition is suitable for use to stimulate the adaptive immune response to the antigen component in the subject with minimal side effects.

The present invention relates to the design and screening of effective flagellin-conjugated vaccines by utilizing a panel of genetic markers. The methods of the present invention may be used to determine whether the TLR5 signalling properties of flagellin-conjugated vaccines are maintained after fusion of antigen to flagellin.

### BRIEF DESCRIPTION OF THE FIGURES

FIG. 1. A schematic representation of full length hemagglutinin (HA1) and truncated versions of HA, HA 1-2, HA 1-1 and HA1s.

FIG. 2. A schematic representation of three different HA vaccine formats (C-term, R3 and R3.2x). D0, D1, D2 and D3 are the four domains of flagellin and the genetically fused antigen is encircled. The primary TLR5 binding site is located in D1.

FIG. 3. A diagram illustrating the induction of IL-6 cytokine gene expression/responses individual mice by different flagellin based vaccines and controls, namely Flagellin is the native flagellin protein alone, HA1-2PR8 is the HA1-2 globular head of the PR8 HA1 strain, R23 is a specific construct with a B strain HA globular head, Potent is a collection of HA-flagellin constructs for HA1 and HA5 strains that have previously been very potent in testing, Poor is a collection of HA-flagellin constructs include R3 and C-terminus for the B strain have previously been poorly potent in testing, and completely dead is a HA-flagellin construct that previous had no potent in previous testing

FIG. 4. A schematic representation of the orientation of the globular head domain of HA relative to flagellin in two constructs.

FIG. 5. A diagram illustrating the neutralizing titers for R3 and R23 B Florida constructs with varying linker regions. The cutoff for positive responses is depicted by the dotted line. HA (full length) refers to a full length HA protein expressed in a Baculovirus expression system.

FIG. 6. A diagram illustrating HAI titers for H3 Perth constructs comprising longer globular heads of either HA1 or HA1s.

FIGS. 7A and 7B. Space filling models of flagellin and hTLR5, respectively, demonstrating the complementarity of charge distribution on their respective surfaces.

FIGS. 8A, 8B and 8C. A list of genes whose expression levels that may be useful in determining immunogenic compositions with enhanced immunogenicity and/or reduced reactogenicity.

FIG. 9. A diagram illustrating the expression of selected genes when mice were injected with 1 μg STF.HA1-2 PR8 at timepoints 3 h, 6 h and 10 h.

FIG. 10. A diagram illustrating the expression of selected genes when mice were injected with 1 μg STF.HA1-2 HA1-2 SI, STF24XM2e, flagellin, STF2R3HA1-2 BFlo, and HA1-2PR8 at 3 h.

FIG. 11. A diagram of a mouse HAI assay for several vaccine candidates.

FIGS. 12A, 12B and 12C. Diagrams for evaluating reactogenicity of the I411A mutation in STF2 HA1-2 SI construct.

FIGS. 13A, 13B, 13C and 13D. A diagram illustrating the expression of selected genes when mice were injected with 1 μg wild-type C-term SI vaccine and the I411A mutation at 0.1 μg, 0.3 μg, 1.0 μg and 3.0 μg.

FIGS. 14A, 14B, 14C, 14D, 14E and 14F. A diagram comparing fold rise for several genes markers for various doses of wild-type C-term SI vaccine and the I411A mutation.

### DEFINITION OF TERMS

“Fusion protein” as used herein refers to a protein generated from at least two distinct components (e.g. a protein portion of HA and a flagellin). Fusion proteins can be generated recombinantly or chemically.

“A portion of a protein” or “protein portion” as used herein in reference to a naturally occurring viral hemagglutinin, refers to any part of the naturally occurring viral hemagglutinin that is less than the entire naturally occurring hemagglutinin.

“A globular head” as that phrase is used herein, refers to a portion of a protein of a naturally occurring viral hemagglutinin that includes the receptor or sialic acid binding regions.

“Flagellin control sample” as used herein, refers to naturally occurring flagellin, full-length flagellin or a portion of flagellin that elicits an immune response equivalent to full-length flagellin.

“HA1-1” as used herein, refers to a protein portion of a viral hemagglutinin that includes at least one β-sandwich that includes the substrate binding site, which includes at least about two β sheets, at least about two to about three short α-helixes, at least one small β sheet and at least one additional small β sandwich at the bottom of the molecule and at least about four disulfide bonds. The β sandwich that includes the substrate binding site of the HA1-1 includes about four β-strands as the bottom sheet. At least about one a helix of the HA1-1 portion is located by the side of the β sandwich that includes the substrate binding site and at least about one to about two are located at the bottom of the β sandwich that includes the substrate binding site. The small β sandwich of the HA1-1 can include at least about two to about three β-stands in each β sheet; or about three to about four β-strands.

“HA1-2” as used herein, refers to a protein portion of a viral hemagglutinin that includes at least one β-sandwich that includes the substrate binding site, at least about two to about three short α-helixes, at least one small β sheet at the bottom of the molecule and at least about two disulfide bonds. A β-strand in a viral hemagglutinin can include between about two to about 15 amino acids. A small β-sheet can include from about two to about three β-strands. The β-sandwich that includes the substrate binding site of HA1-2 can further include at least about four β-strands as a top sheet and at least from about three to about four β-strands as the bottom sheet.

### DETAILED DESCRIPTION OF THE INVENTION

The immune system includes two interconnected branches comprising innate and adaptive immunity. Innate immunity is the more ancient of the branches and is essentially, a rapid response and containment system. It cells use invariant receptors to detect and signal the occurrence of microbial infection. These signals serve two extremely important functions: they initiate an inflammatory cascade that helps contain the infection and they activate the adaptive immune response. The adaptive immune response is tailored to the infection at hand. It is based on the expansion of pathogen-specific lymphocytes via a process that is highly effective but takes days to fully develop. The inflammation that is typically associated with the early stages of microbial infection (i.e., redness, heat, swelling, and pain) therefore, is actually initiated by innate immune recognition. Cells of the adaptive immune response can contribute to and exacerbate these effects, but the primary signals that start the inflammatory response and ultimately resolve it are linked to innate immune recognition. Recognition of microbes by the innate immune system can trigger an inflammatory response for containment of infection and orchestration of adaptive immunity.

Innate immune receptors are often referred to as pattern recognition receptors (PRRs) and signaling through these receptors is what links the recognition of microbes to the induction of an inflammatory response. The best characterized PRRs are the Toll-like receptors or TLRs which recognize conserved components (known as pathogen associated patterns or PAMPs) or bacteria, viruses, fungi and protozoans. Recognition activates the cell and initiates a conserved signaling cascade that leads to the activation of NF-κB and IFN-regulatory factor (IRF) transcription factors. These transcription factors in turn drive the expression of pro-inflammatory genes (such as TNF-α and IL-1) and this leads to the production of the signals that initiate inflammation and orchestrate adaptive immunity. In addition to the membrane-spanning TLRs, a large family of cytosolic PRRs participates in the detection of pathogens that are able to enter host cells. In general, the membrane-spanning and cytosolic PRRs are thought to serve complementary functions in the inflammatory process but there is now some evidence of redundancy in these systems in that the same PAMP can trigger both a TLR and a cytosolic PRR.

An effective inflammatory response needs to be rapid and appropriate for the pathogen at hand but also self-limiting in terms of the damaging aspect of inflammation. It is a highly regulated process aimed at insuring the appropriate degree of inflammation. If regulated improperly the inflammatory components have the capacity to cause extreme damage to the host. Yet, at the same time, the innate inflammatory response is also intricately linked to the adaptive immune response and many of the signals important to inflammation are also important to efficient lymphocyte activation. These signals, therefore, must be sufficient for initiation of the adaptive response. Determining the appropriate degree of inflammatory response is a delicate balance between insuring that the signals are sufficient but appropriately limited.

Given that the signals for inflammatory and adaptive responses are intimately shared, a common view is that the magnitude of these responses will parallel each other. More specifically, it is believed that a reduction in the inflammatory response will lead to a parallel reduction in the adaptive immune response. Typically there is a parallel dose relationship in response to an immune stimulus, whether it be the whole pathogen or a vaccine based on a PAMP, and the inflammatory and adaptive responses that develop. More recently, it has been demonstrated that it is possible to induce an adaptive immune response in the absence of the robust systemic cytokine production typically associated with potent innate immune stimulation. As described herein, if an innate pathway is “knocked-out” leaving the possibility open that the innate immune stimulation is still operating via another redundant pathway and supplying additional signals other than the cytokines Proinflammatory cytokines produced in response to PAMPs may be in excess of what is required to induce an effective adaptive immune response. So while there has been some suggestion that that is may be possible to separate the inflammatory component from the adaptive component of an innate stimulus response, mechanisms for independently controlling activation of inflammatory and adaptive responses have not been identified. Further, the threshold of innate immune stimulation required to support development of an adaptive immune response is not known; and give the complex, multi-faceted nature of the innate immune response this threshold likely could derive from multiple, different combination of signals.

Flagellin is a PAMP that can interact with the TLR5 receptor as well as with at least two cytosolic PRR receptors. Genetically modified forms of flagellin have been produced which differentially affect reactogenicity (the outcome of an inappropriately exaggerated inflammatory response) and adaptive immunity. Some forms retain the ability to potentiate an immune response while significantly lowering reactogenicity. Portions of flagellin are evaluated for reactogenicity, immunogenicity and TLR5 signaling, which can be identified as at least two classes of alterations. One class reduces TLR5 signaling to a level that maintains immunogenicity while reducing reactogenicity. A second class reduces the ability of the flagellin molecule to trigger cytosolic PRR. Thus, it is possible to separate the inflammatory and adaptive components of the immune response potentiated by flagellin. For the TLR5 based mechanism there is a minimum threshold of signaling that permits the initiation of an immune response in the absence of a reactogenic (exaggerated inflammatory) response.

Conjugation of flagellin to a vaccine antigen is a way to make the vaccine more immunologically potent and therefore effective. Flagellin works by binding Toll-like receptor 5 (TLR5) present on cells of the innate immune system. TLRs recognize certain ‘patterns’ that are conserved in flagellin. Binding of flagellin to the TLR5 receptor triggers a series of innate and adaptive immune responses that are necessary for orchestration of an effective immune response. A key initial event that follows binding to TLR5 is the propagation of a signal to the nucleus of the immune cell. This signaling event leads to the differential regulation of key genes and the upregulation of cell surface and secreted proteins that are required to initiate an immune response.

Conjugation of antigens to flagellin to form vaccine compositions risk alteration or destruction of the patterns within the flagellin. This may occur as a result of interactions among the amino acids of the antigen with the amino acids of the flagellin which may result in a loss of TLR5 binding activity. To determine whether an antigen has been successfully conjugated to flagellin, presently animals are immunized with the conjugate, followed by a booster dose (2-3 weeks after initial dose), after which sera is harvested (2-3 weeks after booster dose) for in vitro assays or to conduct challenge studies in immunized animals. The current process involves considerable time, resources and expense.

The present invention relates to identification of a set of gene products and/or serum cytokines that are made in response to flagellin and flagellin-conjugated vaccines. These genetic markers and cytokine levels may be used to determine whether a particular flagellin containing composition such as flagellin fusion vaccines will be more or less potent or more or less immunogenic or more or less reactogenic than other flagellin containing compositions. The majority of genetic markers disclosed in the present invention are made in response to flagellin as well as flagellin containing compositions such as flagellin-antigen fusion proteins. These markers appear in the spleens of Balb/c mice as early as 1 hour following administration of a vaccine. A subset of genetic markers is associated with downstream signaling events and may reflect the initial stage of organizing an adaptive immune response. Thus, identification of a set of gene products that are made in response to flagellin and flagellin-conjugated vaccines can be used to guide the optimization of a given antigen/flagellin fusion protein structure for immunogenicity and reactogenicity. The invention also relates to methods of assessing suitability of a composition that includes at least one flagellin component and at least one antigen component for use to stimulate an adaptive immune response to the antigen component in a subject with minimal side effects, by measuring expression of genes and/or serum cytokine levels including but not limited to Ccl2, Cd80, Csf2, Cxcl10, Fadd, Hspa1a, IL-1a, IL-1b, IL-6, Irf3, Mapk8ip3, Nf-kb2, Nf-rkb, Ppara, Eif2ak2, Tbk1, T1r2, T1r3, T1r4, T1r7, Tnf, Tnfaip3, Traf6, Jun, Cebpb, Fos, Ptgs and Ticam and IL6 and TNF respectively in a test sample wherein a defined minimum & maximum expression of the genes and cytokines in the test sample relative to a flagellin control sample indicates that the composition is suitable for use to stimulate the adaptive immune response to the antigen component in the subject with minimal side effects.

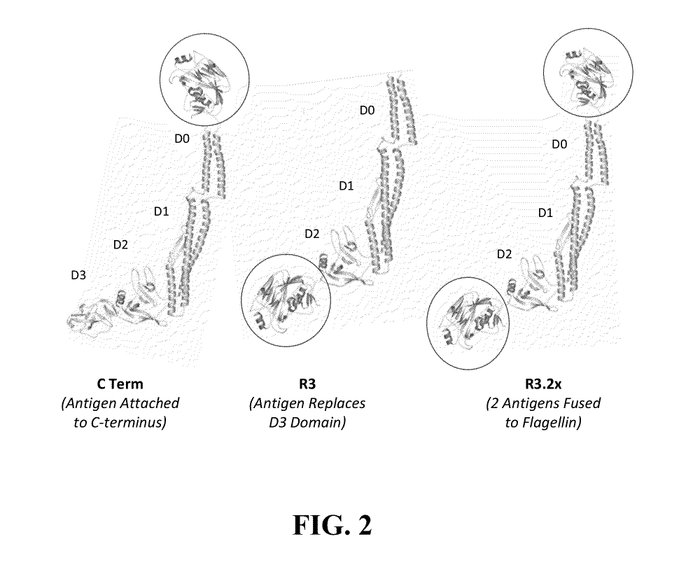

Vaccine compositions utilizing flagellin fused (in combination) with one or more antigens which differ in the attachment site of the antigen to flagellin are shown in FIG. 2. Some formats carry two copies of the antigen. C-terminal format type vaccines (C term) genetically fuse the vaccine antigen to the C terminus of flagellin. R3 format vaccines replace domain 3 by genetically fusing the vaccine antigen to D2, R3.2x format vaccines fuse one copy of the vaccine antigen to the C terminus while an additional copy of the antigen replaces domain 3. The fusion proteins comprising a flagellin and at least one antigen can include a linker between at least one component of the fusion protein (flagellin) and at least one other component of the fusion protein (e.g. HA1-1, HA1-2) or any combination thereof “Linker” as used herein in reference to a fusion protein refers to the connector between components of the fusion protein in a manner that the components are not directly joined. Fusion proteins can include a combination of linker between distinct components of the fusion protein similar or like components of the fusion protein. The linker can be an amino acid linker which can include naturally occurring or synthetic amino acid residues. The amino acid linker can be of various lengths and compositions. Each of these different vaccine formats has different properties. More specifically, the attachment point, or location, of the antigen relative to flagellin can influence the antigenic, the immunogenic and even the reactogenic properties of the vaccine.

Antigens that can be used in combination with flagellin in the compositions and methods of the present invention are any antigen that will provoke an immune response in a human. Antigens used in the compositions of the present invention include viral antigens such as influenza viral antigens (e.g. hemagglutinin (HA) protein, matrix 2 (M2) protein, neuraminidase), respiratory synctial virus (RSV) antigens (e.g. fusion protein, attachment glycoprotein), papillomaviral (e.g. human papilloma virus (HPV), such as an E6 protein, E7 protein, L1 protein and L2 protein), Herpes Simplex, rabies virus and flavivirus viral antigens (e.g. Dengue viral antigens, West Nile viral antigens), hepatitis viral antigens including antigens from HBV and HC. Antigens used in the compositions of the present invention include bacterial antigens including those from Streptococcus pneumonia, Haemophilus influenza, Staphylococcus aureus, Clostridium difficile and enteric gram-negative pathogens including Escherichia, Salmonella, Shigella, Yersinia, Klebsiella, Pseudomonas, Enterobacter, Serratia, Proteus. Antigens used in the compositions of the present invention include fungal antigens including those from Candida spp., Aspergillus spp., Crytococcus neoformans, Coccidiodes spp., Histoplasma capsulatum, Pneumocystis carinii, Paracoccidiodes brasiliensis, Plasmodium falciparum, Plasmodium vivax, Plasmodium ovale, and Plasmodium malariae. In preferred embodiments the antigen contained within the compositions of the present invention is an antigen from influenza virus. A preferred antigen is hemagglutinin (HA). In preferred embodiments the HA sequences are conjugated to flagellin or to engineered flagellins as described in WO 2009/128950 herein incorporated by reference.

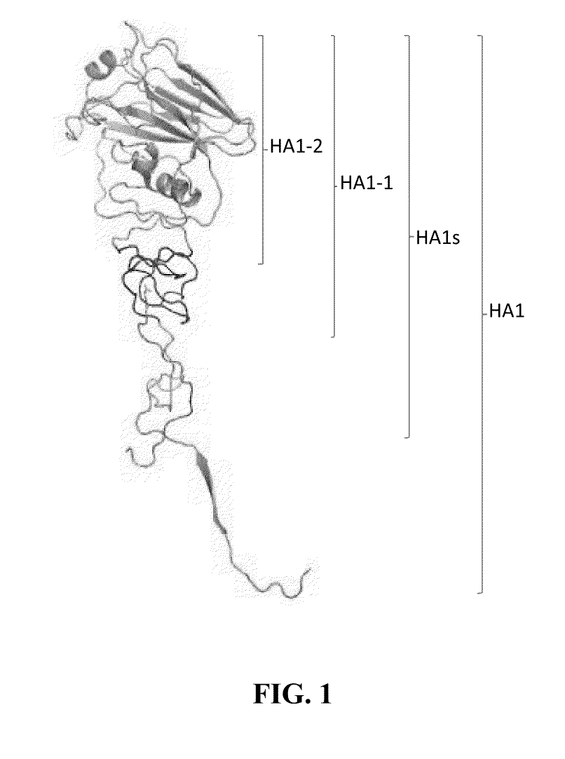

FIG. 1 shows a ribbon diagram of an HA antigen from influenza. HA-1 is the full-length hemagglutinin and HA1-1, HA1-2 and HA1-s are truncated versions of the full-length HA. Genetic fusions of the HA1-2 or HA1-1 HA subunit of influenza virus have been constructed for several different subtypes of influenza. HA1-1 and HA1-2 are further described in WO 2007/103322, herein incorporated by reference. HA1-1 and HA1-2 subunit from multiple HAs of the H1 and H5 subtypes to flagellin are highly immunogenic and efficacious in preclinical challenge models. Differences in immunogenicity and reactogenicity may be based on the placement of the antigen subunits relative to the flagellin. For example, an R3 format of some hemagglutinin antigens from certain influenza strains appears to be more immunogenic than the C-terminal format. R3 and R3.2x formats of a other hemagglutinin antigens elicit higher HAI titers than the C-term constructs. R3 and R3.2x format vaccines may be less reactogenic than C-term formats. Furthermore alterations in the amino acid sequences of the vaccines may also alter the immunogenicity and/or reactogenicity of the vaccine constructs (See Examples). For influenza B presentation of the HA subunits in a C term or R3 vaccine format are often are poorly immunogenic. Specifically, C term forms of influenza B Florida HA have not elicited protective immune responses in naïve mice nor do they appear to boost pre-existing titers in primed mice. R3 forms of influenza B Florida vaccines only elicit measurable immune responses in animals that have already received an immunization of commercial vaccine (primed animals). The disulfide bonds of all construct formats are properly formed and the antigen reacts well with antibodies specific for the antigen, indicating that the antigen has folded properly. Thus, the lack of activity appears not to be related to improper folding of the HA antigen.

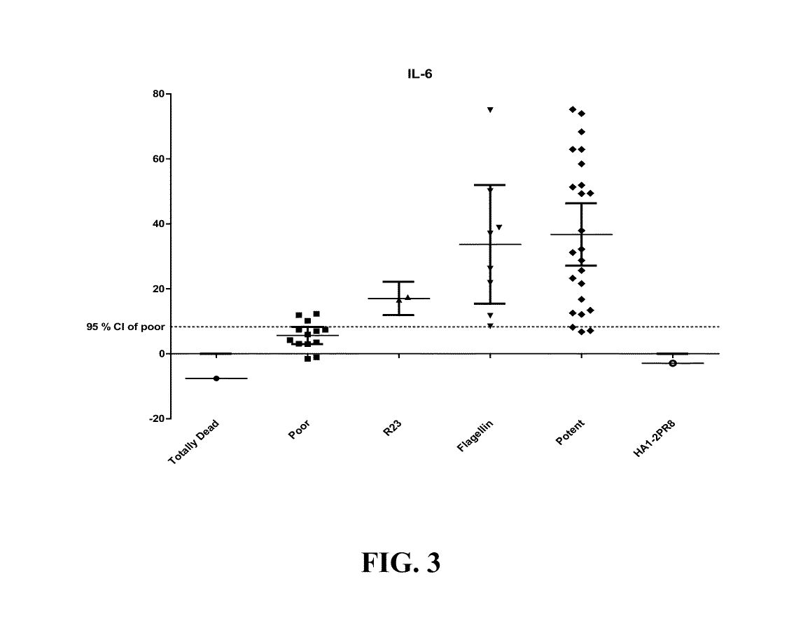

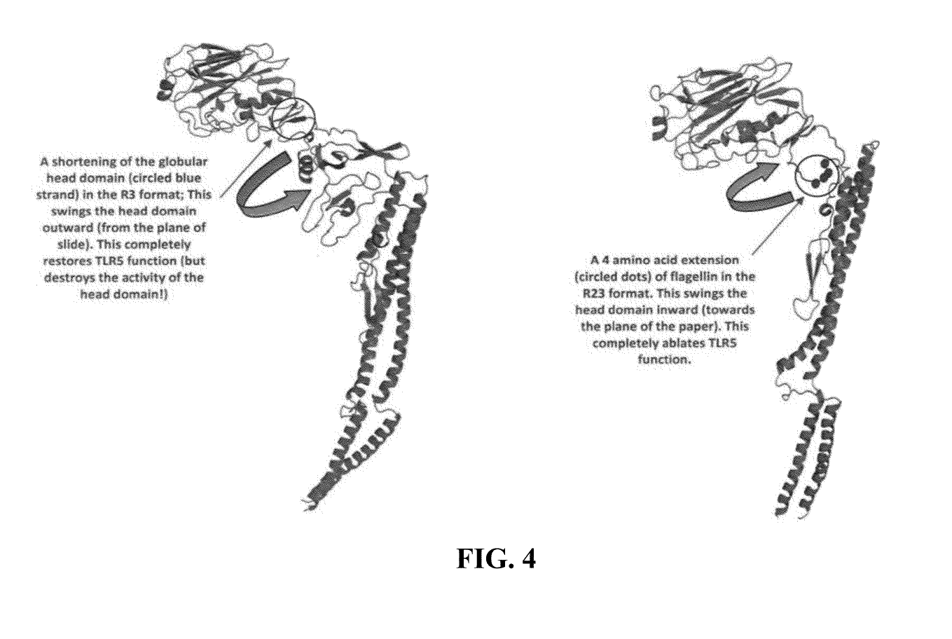

There are a set of genes and cytokine proteins, along with thresholds for their production, that appear to be important for the immune potentiation function of flagellin. These measures can be used as guides to the design of immunogenic vaccines. Using these measures we find that the R3 format of B Florida vaccine fails to trigger TLR5 responsive genes following immunization while the R23 format triggers low level up-regulation of these genes (see FIG. 3). The fold regulation is reported on the y-axis. Completely inactive and vaccines lacking flagellin (HA1-2 PR8) have been used as controls for setting a threshold for the extent of TLR5 stimulation that is required for immunopotentiation. The upper 95% confidence interval of the fold regulation by the control vaccines serves as the lower limit of stimulation required. R3 based influenza B vaccines are represented by the ‘poor’ category of vaccines. R23 forms cross over this threshold and therefore represent an incremental improvement for TLR5 signaling.

Various studies indicate that less than optimal construct design resulting in poor TLR5 agonist activity rather than misfolding of the antigen is the basis of the poor immunogenicity of influenza B constructs. A panel of new constructs using flagellin and HA from influenza B which alters the sequences linking the globular head to flagellin have been designed and constructed. R3 constructs as described above were designed and made as well as a new R23 form in which the D2 and D3 domains of the flagellin were replaced with HA antigen. Different linkers connecting the flagellin to the antigen alters the relationship between the antigen and the flagellin, such as the distance of the antigen head domain from flagellin, the angle of the antigen head domain in relation to flagellin and the chemistry of the linking sequences. The constructs were screened for TLR5 activity (functionality of the flagellin moiety) and in vitro inhibition of neutralization (functionality of the antigen moiety). Two constructs were particularly informative. Construct HL300 had 14 residues deleted from the N terminus of the HA globular head domain. This deletion shortens the N terminal linking region to flagellin and serves to swing the head domain outward, away from flagellin. This alteration completely restored TLR5 function in the in vivo TLR5 assay. However, deletion of these 14 residues from the HA moiety had a negative impact on the antigenicity of the HA globular head. A second construct added 4 amino acids to the N terminal linking region. This extension of the N terminal linker to flagellin swings the globular head domain in the opposite direction and ablated TLR5 function. Thus, the orientation of the globular head domain relative to flagellin can impact the antigenicity and/or the ability of the construct to trigger TLR5 responses. The effect may be more pronounced for influenza B and H3 subtypes. By altering the sequences that genetically link the vaccine antigen to flagellin either with respect to length and/or charge or polarity the spatial orientation of the antigen to the flagellin can be manipulated to further modify or improve the immunogenicity of flagellin-antigen fusion vaccines. Importantly, the optimal linking sequences can differ for the different influenza subtypes. Thus, the length of the antigen, the vaccine format and the fusion linkers can be used together to create vaccines with desired immunogenic and reactogenic properties. In particular, the length of the linkers linking the flagellin to the antigen may be altered so that the antigen does not interact or block the TLR5 binding site on the flagellin. Alternatively or in addition, the charge of the linkers may also be altered such that the antigen does not interact or block the TLR5 binding site on the flagellin. Thus, the present invention provides methods for optimizing the antigenicity (ie. increasing the antigenicity) and/or the reactogenicity (ie decreasing the reactogenicity) of flagellin-antigen fusion proteins including flagellin-HA fusions that have been poorly immunogenic and highly reactogenic by altering the orientation of the flagellin to antigen

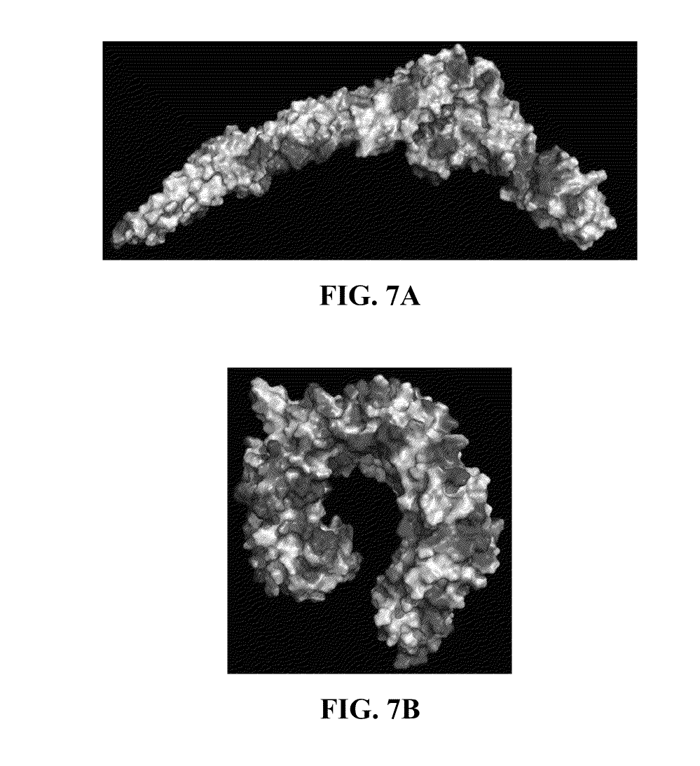

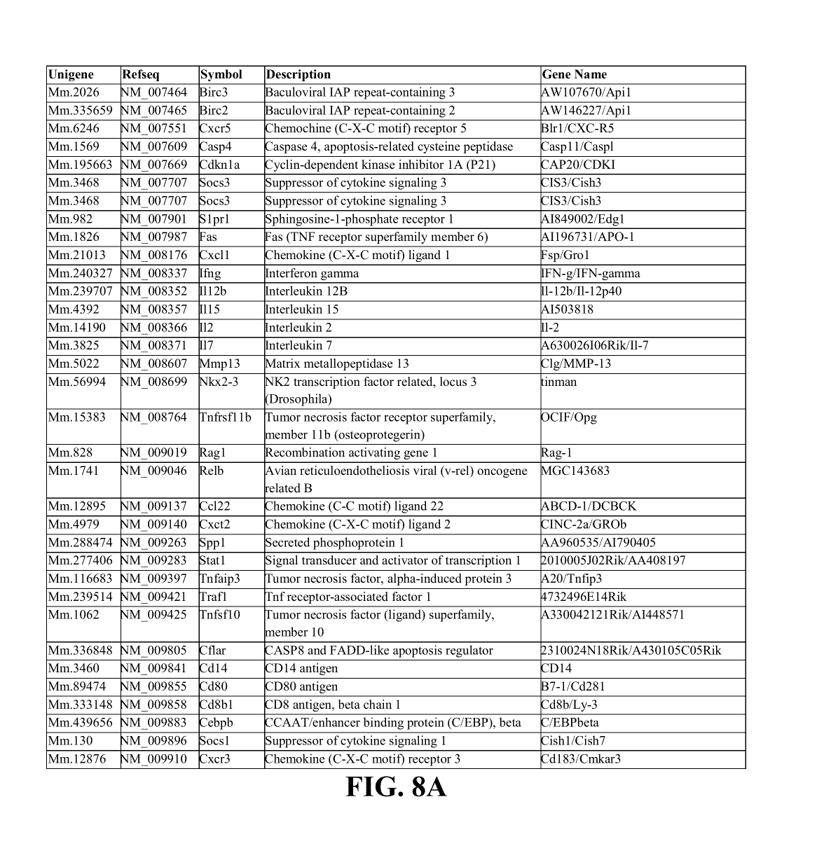

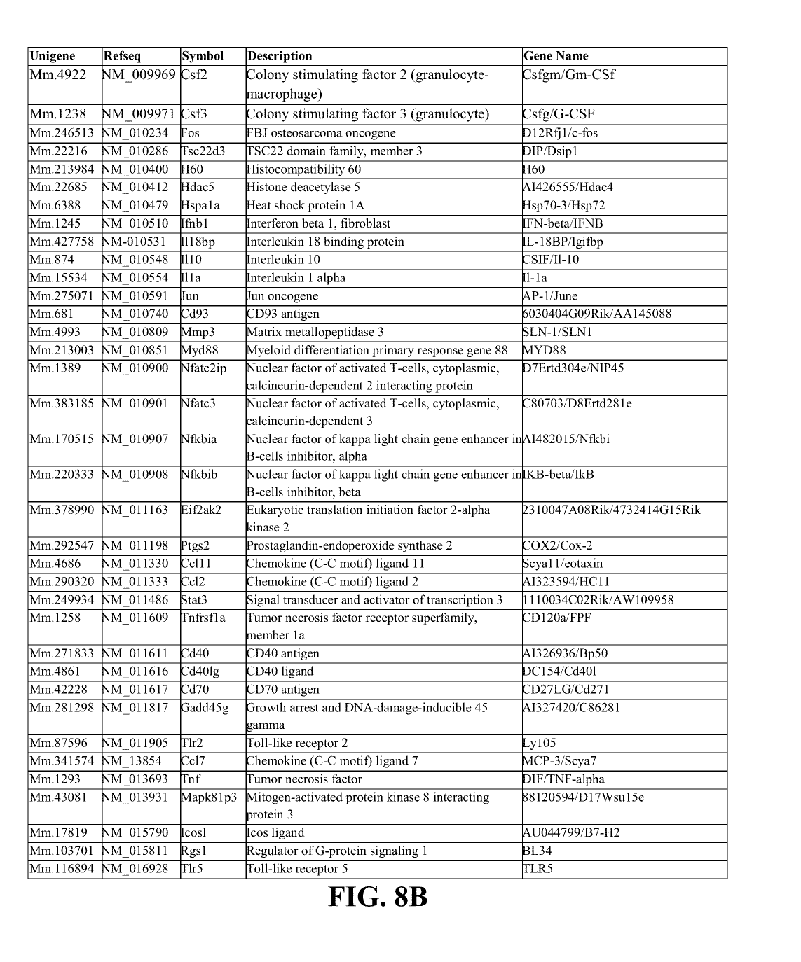

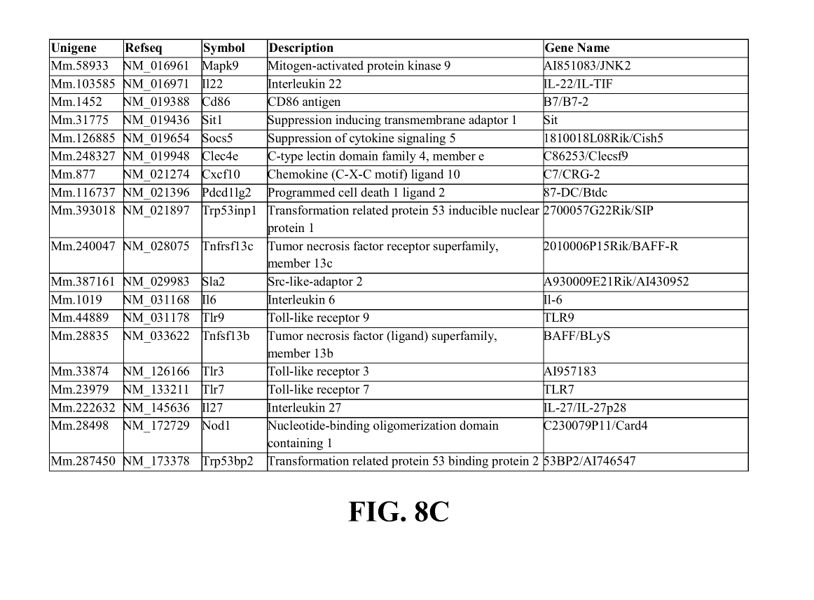

Sequences in D0, D1 and D2-D3 are known to contribute to TLR5 binding. Structural models of TLR5 have identified 3 regions, or charged patches, which spatially match 3 oppositely charged patches on flagellin and could be involved in flagellin/TLR5 binding. One of these regions is clearly known to be required for TLR5 binding. The other 2 are in regions of domains 0 and 2 and are thought to contribute either to binding and/or to flagellar filament assembly and motility. As can be seen in FIGS. 7A and 7B flagellin appears to have three charged patches in a triangular array which are the mirror image of the same triangle in the hTLR5 model in which three opposite charged patches spatially correspond to those in flagellin. The active form of hTLR5 may be a dimer and according to space filling models the charge complementarity of flagellin and hTLR5 is replicated on their reverse surfaces. If the spatial orientation or charge distribution is disturbed the antigen may block the hTLR5 binding site on the flagellin in various ways including sterically interfering with the binding site or interfering with the binding site on the flagellin or on the TLR5 due to charge complementarity. In such cases the TLR5 binding site might be blocked and/or the globular head of the antigen might be inaccessible or improperly folded. For example, constructs carrying high pI globular head domains in the R3 position, such as B Florida, would replace one of these regions with the wrong charge and thus hinder interaction with TLR5. Linkers that include a negative charge, however, such as that carried by the HL352 linker (See Example 1) provide an appropriately charged alternative region for binding.

Multiple HAs of the H3 subtype genetic fusions of the HA1-2 HA subunit presented in the standard flagellin formats are poorly immunogenic. Specifically, C term and R3 forms of H3 Wisconsin, H3 Aichi and H3 Perth HA1-2 HA fail to elicit protective immune responses in naïve mice or boost pre-existing titers in primed mice. Key contributors to the poor immunogenicity may include: 1) H3 HAs, similar to influenza B HAs, have a high pI associated with the globular head. This could interfere with TLR5 signaling or promote an unwanted interaction between the HA head and flagellin which has a low pI or 2) the human H3 HA globular head has an extremely hydrophic core, as compared to the generally more active H1 and H5 HA globular heads. This could complicate proper refolding of the molecule. By changing the charge or polarity of the amino acid sequence on the surface of the globular head the negative effects of the high pI could be ameliorated. Alternatively, the amino acid sequence in the core of the globular head could be altered to resemble its avian counterparts which are easier to refold. It is possible that the avian mutation(s) alleviate the extreme degree of hydrophobicity associated with human H3 HA1-2 proteins and thereby facilitate the refolding under lab conditions.

Thus, it is now possible on the basis of this idealized charge distribution and appropriate spatial orientation it is now possible to design flagellin-antigen fusions that take these issues into account and permit the rational design of a fusion that is spatially and electrostatically “correct”. That is, gives the optimal charge and spatial orientation between the antigen and flagellin such that antigenicity is increased and/or reactogenicity is decreased. Furthermore, it is possible to design and screen these flagellin-antigen fusion proteins by utilizing a panel of genetic markers to determine whether the TLR5 signalling properties of flagellin-conjugated vaccines are maintained after fusion of antigen to flagellin. Genetic markers may also be utilized to determine whether the antigen component of the fusion protein can stimulate an adaptive immune response to the antigen in a subject with minimal side effects.

The genetic marker screening combined with the re-design of flagellin-antigen fusion protein constructs can be used to design vaccines with superior immunogenic and reduced reactogenic responses.

The dose of the fusion protein vaccine may be administered to the human within a range of doses including from about 0.1 μg to about 500 μg, 1 μg to about 100 μg, 1 μg to about 50 μg, from about 1 μg to about 30 μg, from about 1 μg to about 25 μg, from about 1 μg to about 20 μg, from about 1 μg to about 15 μg, from about 1 μg to about 10 μg, from about 2 μg to about 50 μg, 2 μg to about 30 μg, from about 2 μg to about 20 μg, from about 2 μg to about 10 μg, from about 2 μg to about 8 μg, from about 3 μg to about 50 μg, 3 μg to about 30 μg, from about 3 μg to about 20 μg, from about 3 μg to about 10 μg, from about 3 μg to about 8 μg, from about 3 μg to about 5 μg, from about 4 μg to about 50 μg, 4 μg to about 30 μg, from about 4 μg to about 20 μg, from about 4 μg to about 10 μg, from about 4 μg to about 8 μg, from about 5 μg to about 50 μg, 5 μg to about 30 μg, from about 5 μg to about 20 μg, from about 5 μg to about 10 μg, from about 5 μg to about 9 μg, and from about 5 μg to about 8 μg. With respect to fusion protein compositions comprising flagellin and influenza antigen the dosage refers to the amount of protein present in the vaccine given to the human. Some of the protein quantity relates to the antigen and some of the protein quantity relates to the flagellin.

The composition can be administered intramuscularly to the human in a single or in multiple doses. The method can further include the step of administering at least one subsequent dose of the flagellin antigen composition to the human.

The immunogenic compositions for use according to the present invention may be delivered as a standard 0.5 ml injectable dose and contain from about 0.1 μg to about 50 μg of antigen. In a preferred embodiment of the immunogenic compositions for use according to the present invention is a standard 0.5 ml injectable dose and contains from about 3 μg to about 20 μg of antigen. The vaccine volume may be between 0.25 and 1.0 ml, suitably between 0.5 ml and 1.0 ml, in particular a standard 0.5 ml. A vaccine dose according to the present invention may be provided in a smaller volume than conventional dosing. Low volume doses according to the present invention are suitably below 0.5 ml, typically below 0.3 ml and usually not less than 0.1 ml.

An “effective amount” when referring to the amount of a composition and a fusion protein administered to the human, refers to that amount or dose of the composition that, when administered to the subject is an amount sufficient for therapeutic efficacy (e.g., an amount sufficient to stimulate an immune response in a subject, an amount sufficient to provide protective immunity in the subject).

The methods of the present invention can be accomplished by the administration of the compositions and fusion proteins of the invention by enteral or parenteral means. Specifically, the route of administration is by intramuscular injection of the composition and fusion protein. Other routes of administration are also encompassed by the present invention including intravenous, intradermal, interaarterial, interperitoneal, intranasal, transdermal, suppositories or subcutaneous routes.

The compositions that include the fusion proteins can be administered alone or as admixtures with conventional excipients, for example, pharmaceutically, or physiologically, acceptable organic, or inorganic carrier substances suitable for enteral or parenteral application which do not deleteriously react with the composition. Suitable pharmaceutically acceptable carriers include water, salt solutions (such as Ringer's solution), alcohols, oils, gelatins and carbohydrates such as lactose, amylose or starch, fatty acid esters, hydroxymethylcellulose, and polyvinyl pyrolidine. Such preparations can be sterilized and, if desired, mixed with auxillary agents such as lubricants, preservatives, stabilizers, wetting agents, emulsifiers, salts for influencing osmotic pressure, buffers, coloring and/or aromatic substances and the like which do not deleteriously react with the compositions administered to the human. Preferred diluents for diluting the vaccines of the present invention include but are not limited to 150 mM NaCl with histidine and trehalose.

The compositions, fusion proteins and proteins of the invention can be administered to a subject on a support that presents the compositions, proteins and fusion proteins of the invention to the immune system of the subject to generate an immune response in the subject. The presentation of the compositions, proteins and fusion proteins of the invention would preferably include exposure of antigenic portions of the fusion protein to generate antibodies. The support is biocompatible. “Biocompatible” as used herein, means that the support does not generate an immune response in the subject (e.g., the production of antibodies).

The dosage and frequency (single or multiple doses) administered to a subject can vary depending upon a variety of factors, including, for example, prior exposure to an infection consequent to exposure to the antigen: health, body weight, body mass index, and diet of the subject or health-related problems. Other therapeutic regimens or agents can be used in conjunction with the methods and compositions, proteins or polypeptides of the present invention.

The composition can be administered to the human in a single dose or in multiple doses, such as at least two doses. When multiple doses are administered to the subject, a second or third dose can be administered days (e.g., 1, 2, 3, 4, 5, 6, 7), weeks (e.g., 1, 2, 3, 4, 5, 6, 7, 8, 9, 10), months (e.g., 1, 2, 3, 4, 5, 6, 7, 8, 9, 10) or years (e.g., 1, 2, 3, 4, 5, 6, 7, 8, 9, 10) after the initial dose. For example, a second dose of the composition can be administered about 7 days, about 14 days or about 28 days following administration of a first dose of the composition that includes the fusion protein.

### EXAMPLES

### Example 1

### Gene Marker Expression for STF.HA1-2 PR8

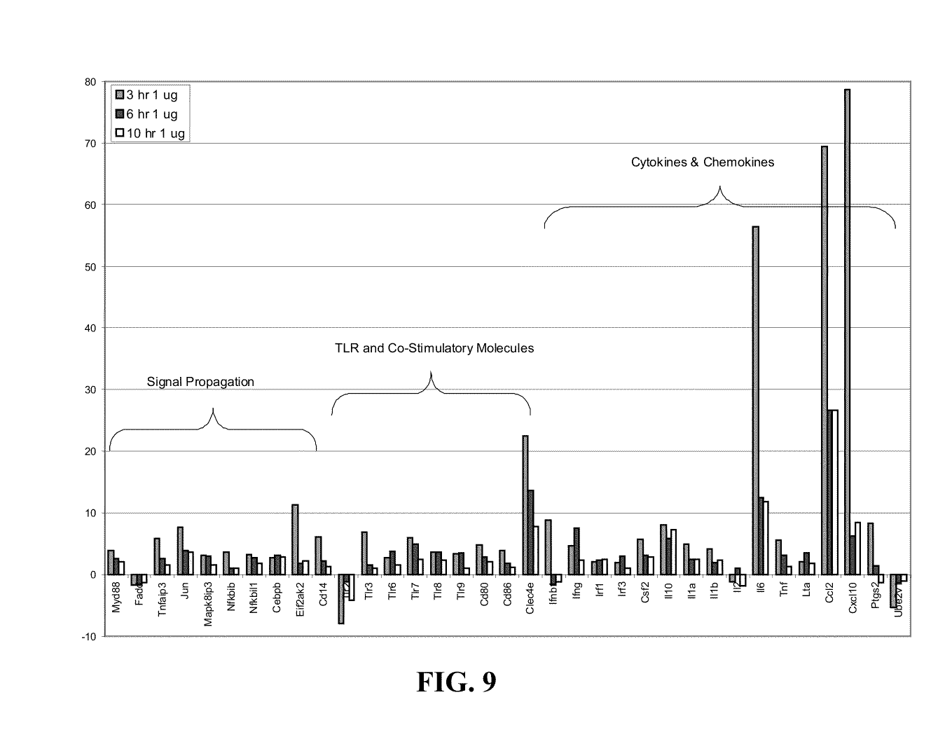

Groups of three Balb/c mice were immunized subcutaneously with 1 μg of STF.HA1-2 PR8, which comprises the globular head domain of A/Puerto Rico/8/34 HA (HA 1-2) fused to the C-terminus of Salmonella typhimurium (type 2) flagellin (STF2). Spleens of the mice were harvested at 3, 6 and 10 hours post-immunization, flash-frozen in a dry ice/ethanol bath. RNA was isolated from the spleens using a Trizol suspension with Purelink RNA minikit (Invitrogen, CA). RNA samples were run on 1% agarose gels to insure the presence of 18S and 28S bands which are indicative of the absence of RNA degradation. Five micrograms of each RNA sample was reverse-transcribed to first stand cDNA, mixed with SYBR green PCR master mix and distributed into a 96 well TLR array PCR plate (SABiosciences). The TLR assay plate contained primer pairs specific for 84 genes that are activated in the TLR signal transduction pathway as well as several control wells which insure the quality of reverse transcription and PCR amplification and which monitor the amount of genomic DNA contamination in the samples. The plate was run on an Applied Biosystems (Carlsbad, Calif.) 7300 Real-time PCR instrument using one denaturation cycle at 95° C. for 10 minutes followed by 40 cycles of 15 second/95° C. denaturation and 1 minute/60° C. annealing and extensions. Data was collected during the 60° C. stage of each cycle. Transcription levels of the genes in response to administration of the vaccine were calculated relative to unimmunized control mice using the DDCt comparative quantitation method. The results of the experiment are shown in FIG. 9.

### Example 2

### Gene Marker Expression for STF2HA1-2 Sl, STF24XM2e, flagellin, STF2R3HA1-2 BFlo, and HA1-2PR8

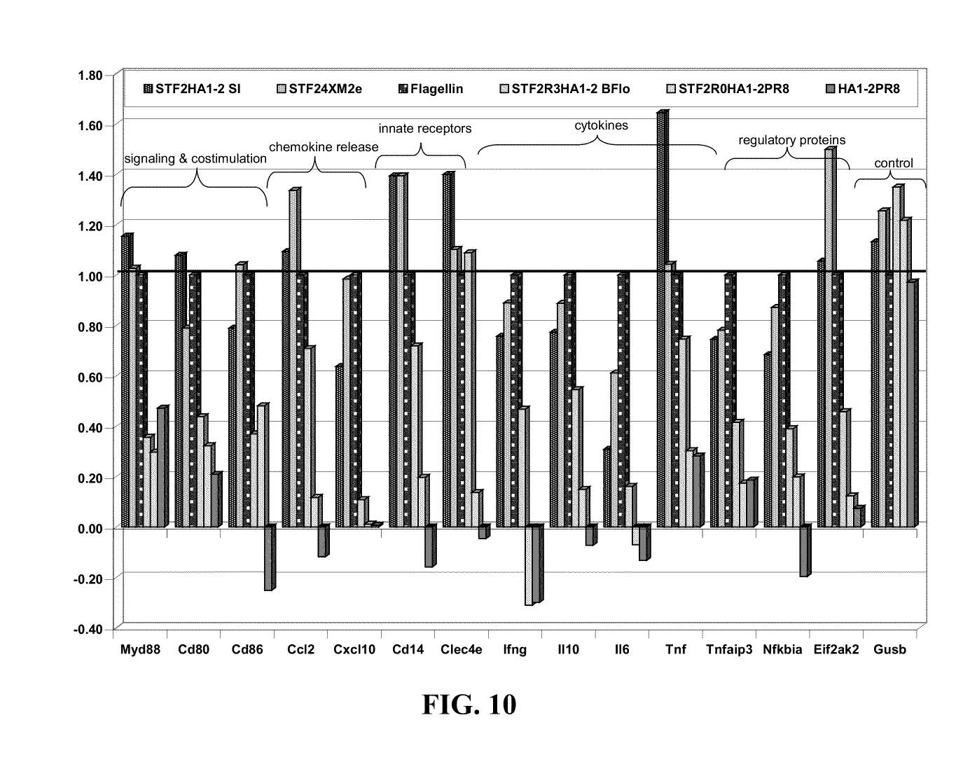

Three groups of Balb/c mice were immunized subcutaneously with 1 μg of STF2HA1-2 Sl, STF24XM2e, flagellin, STF2R3HA1-2 BFlo, and HA1-2PR8. Spleens of the mice were harvested at 3 hours post-immunization, flash-frozen in a dry ice/ethanol bath. RNA was isolated from the spleens using a Trizol suspension with Purelink RNA minikit (Invitrogen, CA). Five micrograms of each RNA sample was reverse-transcribed to first stand cDNA, mixed with SYBR green PCR master mix and distributed into a 96 well TLR array PCR plate (SABiosciences). The TLR assay plate contained primer pairs specific for a subset of genes that are activated in the TLR signal transduction pathway as well as several control wells which insure the quality of reverse transcription and PCR amplification and which monitor the amount of genomic DNA contamination in the samples. Transcription levels of the genes in response to administration of the vaccine were calculated relative to unimmunized control mice using the DDCt comparative quantitation method. The results of the experiment are shown in FIG. 10. In all 15 genes including Fos, Jun, MyD88, Eif2ak2, Nfkbia, TLR3, TLR9 (genes whose products are found in signal transduction), CD80, CD86, CD14 (genes whose products are involved in co-stimulation of immune cells), Ccl2, Cxcl10, IL6, Tnf and Tnfaip (genes whose products activate or recruit cells of the immune system.

### Example 3

### Immunogenicity and Reactogenicity of STF2.HA1-2 Solomon Island (SI) vaccine or a I411A Mutant

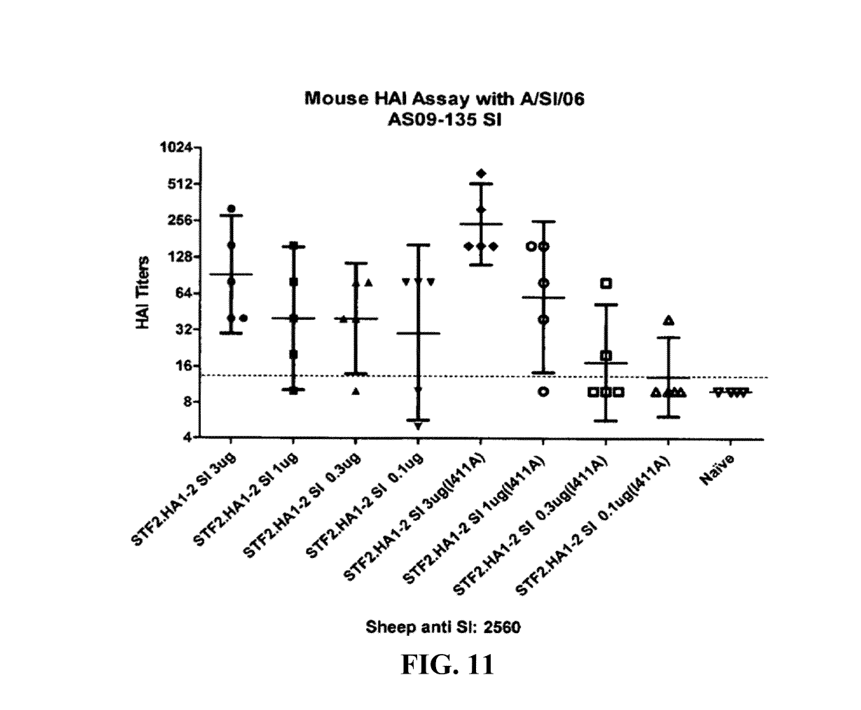

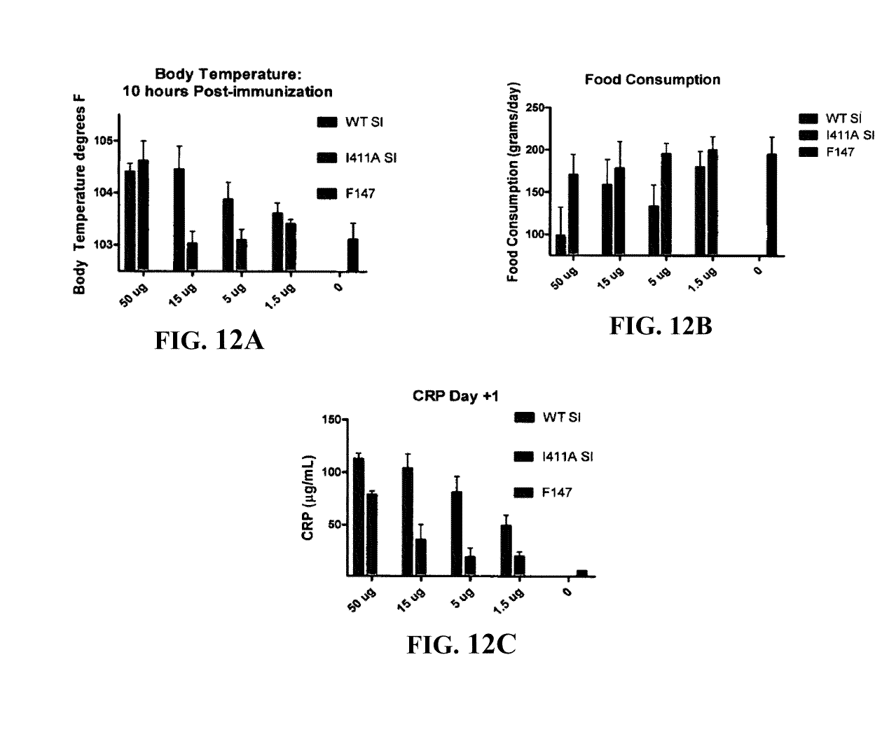

Groups of five Balb/c mice were injected subcutaneously on days 0 and day 14 with either wild-type C terminal STF2.HA1-2 Solomon Island (SI) vaccine or a I411A (an isoleucine to arginine change at residue 411) mutant at a dose of 0.1m, 0.3m, 1.0m, or 3.0m. Serum samples from the mice were subjected to HAI test against A/Solomon Islands 3/2006 virus. Serum samples were treated with receptor destroying enzyme (RDE, Denka Seiken Co., Ltd. Japan) (1 part serum and 3 parts RDE), diluted in a 96-well V-bottom microplate, and incubated with 4 HA units (HAU) of influenza virus in 25 μl for 30 minutes at room temperature. Turkey red blood cells (0.5%) were added (50 μl/well), mixed briefly, and incubated for 30-60 minutes at room temperature. The HAI titers of the serum samples are reported as the reciprocal of the highest dilution at which hemagglutinin is completely inhibited. Sheep anti-influenza serum (NIBSC or CBER) were used as a reference serum to monitor the variability among tests. The results are shown in FIG. 11.

Groups of six rabbits were injected intramuscularly with 1.5 μg, 5 μg, 10 μg, or 50 μg of either wild-type C terminal STF2.HA1-2 Solomon Island (SI) vaccine or a I411A vaccine. F147 formulation buffer (10 mM L-histidine, 150 mM NaCl, 5% trehalose, 0.02% polysorbate 80, 0.1 mM EDTA, 0.5% ethanol, 10 mM Tris, pH 7.2) was delivered alone as a negative control. Rabbit body temperature was monitored prior to vaccination, 10 hours post-vaccination (Day 0) and on two subsequent days. Food consumption was measured from one day prior to vaccination and three day post-vaccination. Rabbits were bled 24 hours after vaccination, serum was prepared and C reactive protein was measured (Immunology Consultants Laboratory, Newberg Oreg.). The results are shown in FIG. 7. Sera from the same rabbit study were also evaluated for neutralizing titers. He results indicate that the immunogenicity of both WT and mutant STF2.HA1-2 SI are comparable in rabbits and mice. However the data in the mouse and rabbit models taken together indicate that the immunogenicity and reactogenicity associated with TLR-linked vaccines are not necessarily related. That is, one vaccine could show high immunogenicity while demonstrating low reactogenicity or low immunogenicity and high reactogenicity.

### Example 4

### Gene Markers related to Immunogenicity and Reactogenicity of STF2.HA1-2 Solomon Island (SI) vaccine or a I411A Mutant

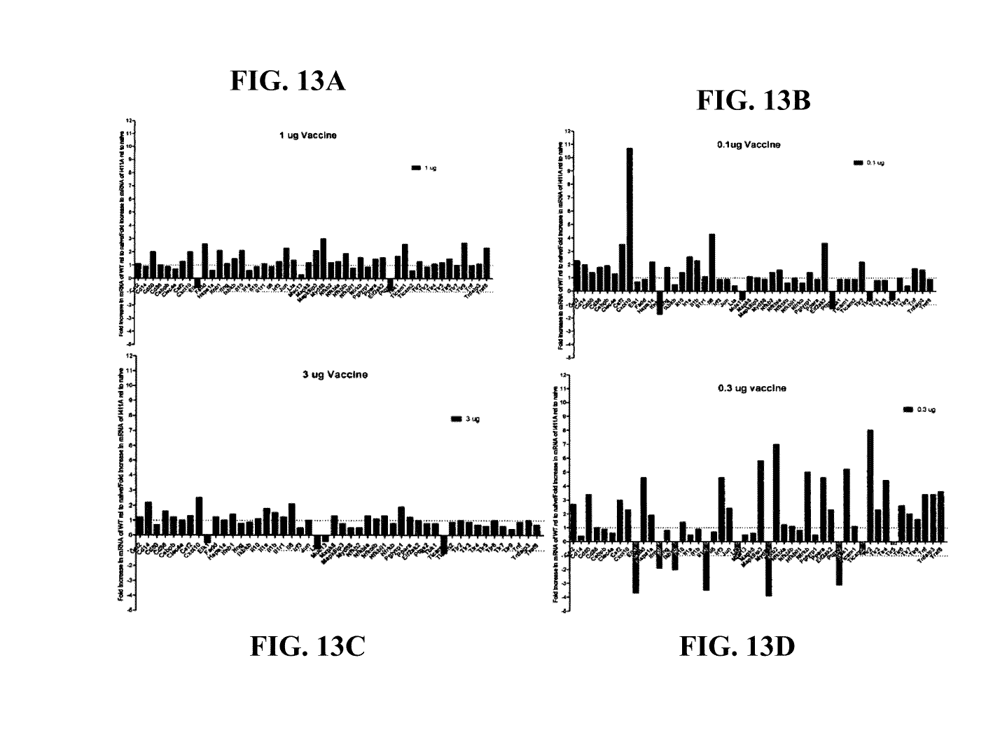

Three groups of Balb/c mice were injected subcutaneously with either wild-type C terminal SI vaccine or an I411A (an isoleucine to arginine change at residue 411) mutant at a dose of 0.1n, 0.3m, 1.0m, or 3.0m. Spleens of the mice were harvested at 3 hours post-immunization, flash-frozen in a dry ice/ethanol bath. RNA was isolated from the spleens using a Trizol suspension with Purelink RNA minikit (Invitrogen, CA). Five micrograms of each RNA sample was reverse-transcribed to first stand cDNA, mixed with SYBR green PCR master mix and distributed into a 96 well TLR array PCR plate (SABiosciences) as described in Example 1. The results are shown in FIGS. 13A-13D in which fold regulation of the wild-type vaccine relative to the unimmunized control was divided by the fold regulation of the mutant vaccine relative to the same control for each gene and expressed as a ratio. A ratio of 1 indicates that the gene was regulated to the same extent following immunization as with either vaccine. Overall, the same genes responded to each vaccine, but to varying extents. For differing doses the fold regulation for many genes trended higher for the wild-type vaccine such as Ccl2, Cd80, Csf2, Cxcl10, Fadd, Hspa1a, IL-1a, IL-1b, IL-6, Irf3, Mapk8ip3, Nf-kb2, Nf-rkb, Ppara, Eif2ak2, Tbk1, T1r2, T1r3, T1r4, T1r6, T1r7, Tnf, Tnfaip3, Traf6, Jun.

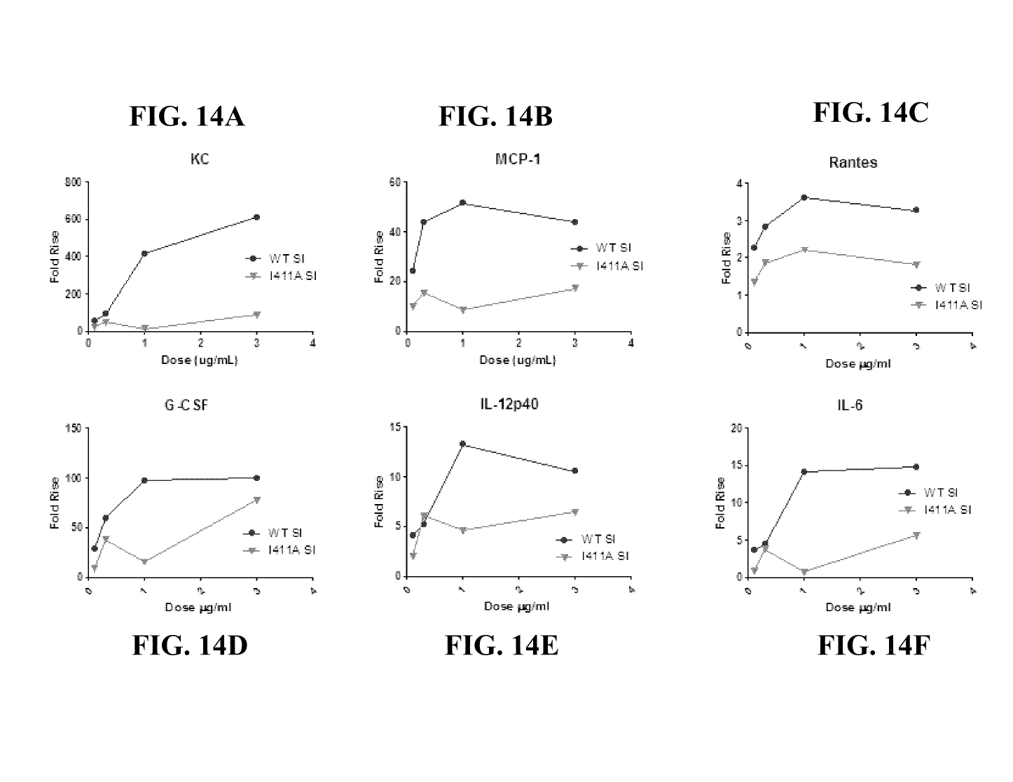

Sera were also harvested and evaluated for pro-inflammatory cytokine levels. At 3 hours post-immunization serum was collected and analyzed for cytokine levels using Bio-Plex mouse 23-plex panel (Bio-Rad, Hercules, Calif.). Samples were diluted at 1:10 in supplied diluent and compared to a standard curve. Values in pg/ml were determined by back-calculating for a 4-parameter logistic fit of the standard curve and then dividing by cytokine levels of the naïve control to produce fold-rise. The results in FIGS. 14A-14F show that in addition to a flatter dose response curve for gene regulation of the I411A substituted vaccine also is associated with a flatter dose response curve for pro-inflammatory cytokine production. A difference in the association constant of flagellin and TLR5 may lead to reduced levels of gene regulation and pro-inflammatory cytokine production.

### Example 5

### Immunogenicity and Reactogenicity of CA07 Vaccine Constructs

Groups of Balb/c mice were injected subcutaneously with 0.1m, 0.3n, 1.0m, or 3.0m of STF2.HA1-2 CA07 vaccine (C terminal format), STF2R3.HA1-2 CA07 vaccine (R3 format) or STF2R32x.HA1-2 CA07 vaccine R32x format). Spleens were harvested at multiple time points post-immunization, RNA prepared and fold regulation of gene expression as compared to naïve mice was determined for a panel of genes as in Example 1. As with the I411A vaccine immunization with any of the vaccines lead to the regulation of the same basic set of genes. The results for R32x construct are similar to the I411A construct in that while the same set of genes is regulated following immunization, several of these genes are not regulated to the same extent as the C terminal format vaccine. Genes that are differentially regulated between R32x and C formats include Cebpb, Cxcl10, Hspa1, IL-6, IL-1, IL-1a, Ptgs, Tnf, Jun, Ticam, Fos and Nfkbia. Genes under-regulated by both I411A and R32x format include Cxcl10, Hspa1, IL-6, IL-1, IL-1a, Tnf and Jun. For both I411A and R32x, the strength of the TLR5 signalling, as measured by the extent of gene regulation for a subset of genes is diminished. Unlike I411A and R32x, the extent of gene regulation is comparable for most genes following immunization of R3 or C-term format vaccines. For a small subset of genes such as IL-1b, TNF and Hspa1a, there is a modest reduction in the extent of gene regulation such that the fold regulation is immediate to that for the C-term format and the R32x formats. However, this may not translate into the level of reduction in cytokine production that was observed for the I411A construct and for R3.2x. The I411A and R3.2x constructs appear to reduce reactogenicity and maintain immunogenicity by lowering the magnitude of the response to TLR5 triggering to a level that is below the threshold of signaling required for reactogenicity yet still above the threshold required for an immune response. The R3 construct appears to achieve a TLR5 signal comparable to C-term but reduces reactogenicity by qualitatively altering an event downstream of TLR5 signalling which may be activation of a cytosolic PRR. The combination of the strength of TLR5 signal and the degree of activation of the PRR predict the range of immunogenicity and reactogenicity: a threshold TLR5 signal may be required for all potent flagellin-linked vaccines, while excess inflammation can be caused by either TLR5 signal that is above the threshold, activation of a cytosolic PRR or both. The present invention relates to the ability to select vaccine candidates which have achieved sufficient TLr5 signal strength to be immunogenic but lack the additional inflammatory signals, either through TLR5 or cystosolic PRR, to be reactogenic.

### Example 6

### Constructs Containing Antigens from H3 and B Subtypes of Influenza

Constructs comprising flagellin and HA antigen of influenza subtype B and subtype H3 were constructed using standard recombinant techniques.

Subtype B Constructs

HL118 R3-2×HA1-2 FL (Add another HA1-2 FL in C-terminal of R3-FL) having a DNA sequence of SEQ ID 1 and protein sequence of SEQ ID 2.
HL163 STF2R3.2xHA1-2 FL (WT) having a DNA sequence of SEQ ID 3 and protein sequence of SEQ ID 4.
HL199 STF2R23.HA1-2 FL (virus WT seq) having a DNA sequence of SEQ ID 5 and protein sequence of SEQ ID 6.
HL261 STF2R23.HA1-2 FL-GSG linkers (WT seq; with GSG linkers between STF2 and HA1-2) having a DNA sequence of SEQ ID 7 and protein sequence of SEQ ID 8.
HL351 STF2R3 HA1-2 FL-1-Loop replace D3 seq (cLoop=12aa replaced 10aa of D3) having a DNA sequence of SEQ ID 9 and protein sequence of SEQ ID 10.
HL352 STF2R3 HA1-2 FL-2-Elongation of C-term (CT; add 9aa in C-term of HA1-2) having a DNA sequence of SEQ ID 11 and protein sequence of SEQ ID 12.
HL354 STF2R3 HA1-2 FL-4-(nGGG+cLGGG+cLoop) having a DNA sequence of SEQ ID 13 and protein sequence of SEQ ID 14.
HL357 STF2R3 HA1-2 FL-7-(nGGG+cLGGG) having a DNA sequence of SEQ ID 15 and protein sequence of SEQ ID 16.
HL384 STF2R3.HA1-2 Sich (B/99)-Corrected having a DNA sequence of SEQ ID 17 and protein sequence of SEQ ID 18.
HL389 STF2R23.HA1-2 FLs (further remove 3aa from C-term half of flagellin) having a DNA sequence of SEQ ID 19 and protein sequence of SEQ ID 20.
HL406 STF2R23 HA1-2 FL (Extend 9aa in C-term of HA1-2) having a DNA sequence of SEQ ID 21 and protein sequence of SEQ ID 22.
HL487 G to S mutation of HL352 (STF2R3 HA1-2 FL-9 aa elongation of C-term) having a DNA sequence of SEQ ID 23 and protein sequence of SEQ ID 24.

Subtype H3 Constructs

HL399 STF2.HA1 Perth (Long HA—add 51aa in N term, 57aa in C-term of HA1-2) having a DNA sequence of SEQ ID 25 and protein sequence of SEQ ID 26.
HL400 STF2R3.HA1 Perth (Long HA—add 51aa in N term, 57aa in C-term of HA1-2) having a DNA sequence of SEQ ID 27 and protein sequence of SEQ ID 28.
HL401 STF2.HA1s Perth (Short version of HL399—N-17aa; C-5aa) having a DNA sequence of SEQ ID 29 and protein sequence of SEQ ID 30.
HL402 STF2R3.HA1s Perth (Short version of HL399—N-17aa; C-5aa) having a DNA sequence of SEQ ID 31 and protein sequence of SEQ ID 32.

### Example 7

### Constructs that Improve Antigenicity, Immunogenicity and/or Reduce Reactogenicity

Flagellin and HA from influenza B fusion constructs were designed, wherein the length and/or the chemical nature of the linker was altered. R3 and R23 forms of flagellin were used. HL352 is an R3 format construct with a 9 aa C terminal extension of the HA globular head region. More specifically, 9 additional amino acids on the C terminus of the B Florida globular head were included in the construct. HL357 also utilized the R3 format; the linkers included the addition of 3 glycines to the N terminal linker and 3 glycines and a leucine to the C terminal linker attaching the HA globular head to flagellin. HL389 is an R23 format vaccine with 3 amino acids of flagellin deleted from the C terminal linker to the globular head.

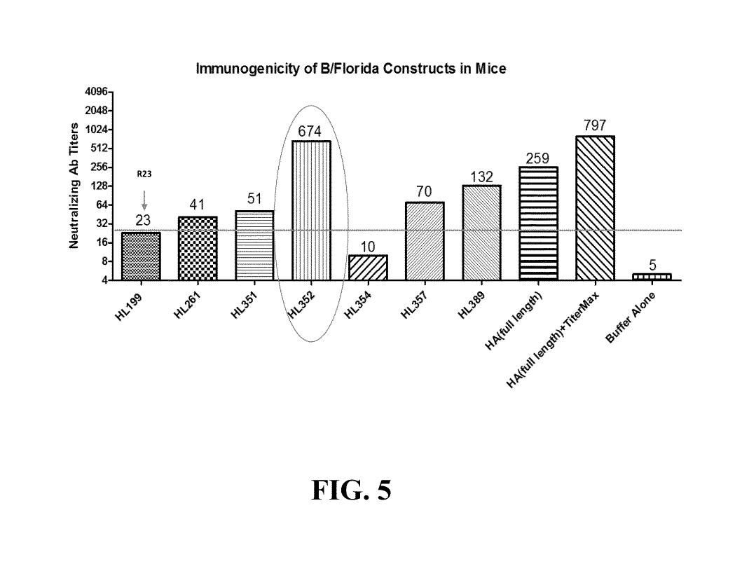

The constructs were screened for in vivo TLR5 activity and antigenicity and the results indicated that each appeared to be potential improvements over R23 (HL199), either by improving TLR5 signaling or by improving the antigenicity. The constructs were also evaluated for the ability to elicit protective (neutralizing) antibody titers in mice (FIG. 5). Three constructs elicited markedly higher titers than the R23 (HL199) construct: HL352, HL357 and HL389. HL352 was as immunologically active as the full length HA protein delivered in the adjuvant Titermax, or more specifically was fully active. (Because the full-length HA contains the entirety of the HA molecule (all potential epitopes), and it is expressed in a eukaryotic system (refolded properly by the cell) it is expected to elicit the maximal immune response possible in the absence of adjuvant. When delivered in adjuvant, it is expected to elicit the maximal response for a recombinant protein.) The modifications to both HL352 and HL357 substantially enhanced the antigenicity of the construct while maintaining a level of TLR5 triggering activity equivalent to R23.

### Example 8

### Manipulations of the H3 Globular Head that Enhance Activity

When R3 and C term HA1-2 H3 constructs are screened for in vivo TLR5 activity and antigenicity, many of the molecules are poorly antigenic but have retained some level of in vivo TLR5 function. These results suggest that improper refolding of the H3 HA globular head is the primary reason for the poor immunogenicity of these H3 constructs.

Amino acid substitutions in the flagellins STF2.HA1-2 or STF2R3.HA1-2 H3 Aichi fusion proteins turn the molecule from being not protective in mouse challenge models to fully protective. One set of these substitutions is in the conserved, extremely hydrophobic core that is characteristic of all human H3 molecules. A three amino acid substitutions ‘loosen up’ this core and make it more ‘H1-like’. Another set of mutations is located on the surface of the protein. Two residues were changed from hydrophobic to charged or polar residues making the surface more hydrophilic. These constructs were tested in mouse challenge models. Results are summarized in Table 1 below.

TABLE 1

| Vaccine Groups | Survivors/total | % survival |
|---|---|---|
| Formulation Buffer | 0/10 | 0 |
| Alone | | |
| STF2R3.HA1-2 Ai 0.4 ug | 0/9 | 0 |
| STF2R3.HA1-2 Ai | 1/10 | 10 |
| STF2R3.HA1-2 Ai 10 ug | 2/10 | 20 |
| STF2.HA1-2 Ai 0.4 ug | 1/10 | 10 |
| STF2.HA1-2 Ai 2 ug | 0/10 | 0 |
| STF2.HA1-2 Ai 10 ug | 0/10 | 0 |
| R3.HA1-2 Ai (3 amino | 8/8 | 100% |
| acid substitutions in the | | |
| core) | | |
| R3.HA1-2 Ai mut-2 | 7/8 | 88% |
| HL368R.001 (2 amino | | |
| acid substitutions on the | | |
| surface) | | |

### Example 9

### Manipulation of Antigen Head Domain Length that Enhance Activity

Constructs which alter the length of the head domain to span HA1s and HA1 (see FIG. 1) have also been designed and evaluated for the ability to elicit protective antibody titers. Re-designed constructs were initially screened for TLR5 activity (functionality of the flagellin moiety), ELISA and in vitro inhibition of neutralization (functionality of the antigen moiety). Mice were immunized, twice, with formulation buffer alone (F 147), full length recombinant HA commercially obtained (HA Perth (PS), HA1 produced by VaxInnate in Baculovirus (HA1 Perth BV053R.001) or C term or R3 forms of HA1 or HA1s fused to flagellin. Commercial TIV (FluV) was included as a positive control. The results show that HA1 and HA1s constructs are able to boost pre-existing titers.

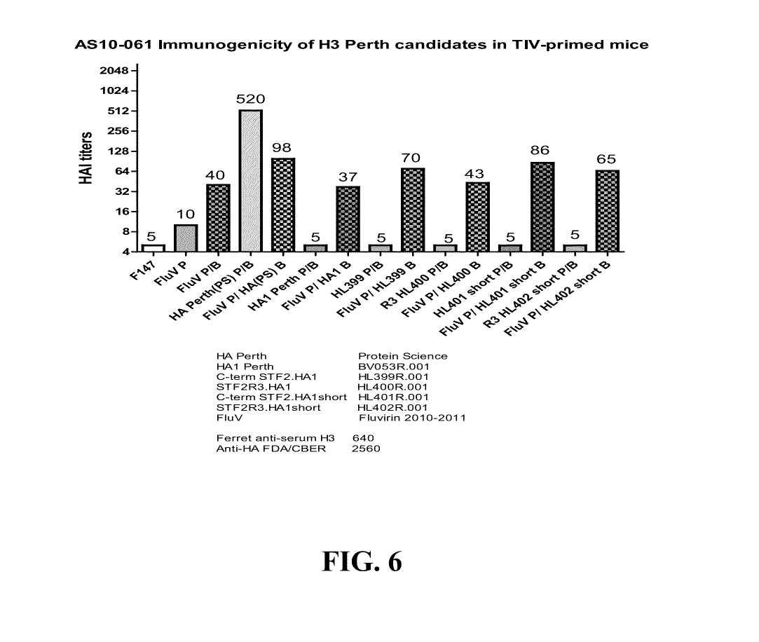

The results showed that R3 and C term forms of H3 Perth that display a longer globular head domain are more active in TLR5 activity assays and antigen specific ELISA than their shorter HA1-2 counterparts. When these molecules were evaluated for the ability to elicit protective antibody titers in mice we find that they are able to boost TIV primed mice but fail to prime and boost (FIG. 6). These results indicate that the longer globular heads are partially active, presumably because the longer globular head refolds better than the shorter HA1-2 subunit.

Within this disclosure, any indication that a feature is optional is intended provide adequate support (e.g., under 35 U.S.C. 112 or Art. 83 and 84 of EPC) for claims that include closed or exclusive or negative language with reference to the optional feature. Exclusive language specifically excludes the particular recited feature from including any additional subject matter. For example, if it is indicated that A can be drug X, such language is intended to provide support for a claim that explicitly specifies that A consists of X alone, or that A does not include any other drugs besides X. “Negative” language explicitly excludes the optional feature itself from the scope of the claims. For example, if it is indicated that element A can include X, such language is intended to provide support for a claim that explicitly specifies that A does not include X. Non-limiting examples of exclusive or negative terms include “only,” “solely,” “consisting of,” “consisting essentially of,” “alone,” “without”, “in the absence of (e.g., other items of the same type, structure and/or function)” “excluding,” “not including”, “not”, “cannot,” or any combination and/or variation of such language.

Similarly, referents such as “a,” “an,” “said,” or “the,” are intended to support both single and/or plural occurrences unless the context indicates otherwise. For example “a dog” is intended to include support for one dog, no more than one dog, at least one dog, a plurality of dogs, etc. Non-limiting examples of qualifying terms that indicate singularity include “a single”, “one,” “alone”, “only one,” “not more than one”, etc. Non-limiting examples of qualifying terms that indicate (potential or actual) plurality include “at least one,” “one or more,” “more than one,” “two or more,” “a multiplicity,” “a plurality,” “any combination of,” “any permutation of,” “any one or more of,” etc. Claims or descriptions that include “or” between one or more members of a group are considered satisfied if one, more than one, or all of the group members are present in, employed in, or otherwise relevant to a given product or process unless indicated to the contrary or otherwise evident from the context.

All publications and patents cited in this specification are herein incorporated by reference as if each individual publication or patent were specifically and individually indicated to be incorporated by reference. The citation of any publication is for its disclosure prior to the filing date and should not be construed as an admission that the present invention is not entitled to antedate such publication by virtue of prior invention.

While this invention has been particularly shown and described with references to example embodiments thereof, it will be understood by those skilled in the art that the various changes in form and details may be made therein without departing from the scope of the invention encompassed by the appended claims.

What is claimed is:

1. An immunologic fusion protein comprising flagellin and an HA antigen linked together by a linker wherein the length of the linker and charge of the linker is optimized to increase immunogenicity and reduce reactogenicity of the fusion protein.

2. The fusion protein of claim 2, wherein the HA antigen is from a B strain of influenza virus.

3. The fusion protein of claim 3, wherein the HA antigen is from a H3 strain of influenza virus.

4. A method of improving the antigenicity of flagellin-antigen fusion proteins comprising optimizing the spatial orientation of the antigen to the flagellin by changing the linker length and/or charge such that a TLR5 binding site on the flagellin is not blocked.

5. A method of improving the antigenicity of flagellin-antigen fusion proteins comprising optimizing the charge distribution of the antigen such that a TLR5 binding site on the flagellin is not blocked.

6. A method of improving the antigenicity of flagellin-antigen fusion proteins comprising decreasing the pI of the antigen such that a TLR5 binding site on the flagellin is not blocked.

7. An immunologic composition comprising a flagellin and an antigen wherein the pI of the antigen has been altered such that the pI of the altered antigen is less than the pI of the unaltered antigen.

8. An immunologic composition comprising a flagellin and an antigen wherein the spatial orientation has been altered to preserve the TLR5 binding site on the flagellin.

9. An immunologic composition comprising a flagellin and an antigen wherein herein the charge on the antigen is altered to such that the antigen does not block the TLR5 binding site.


SEQUENCE LISTING (44 sequences)

SEQ ID NO: 1 - DNA, 2662 bp, Artificial Sequence
  HL118 R3-2xHA1-2 FL (Add another HA1-2 FL in C-terminal of R3-FL)
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagaaagcgtatgatgtgaaagatacagcagtaacaacgaaagctagtactcgtacccgcggtaaactgtgcccagattgcctgaactgcaccgacctggacgtagcactgggccgtcctatgtgcgtgggtactacgccgtctgcaaaagctagcattctgcacgaggttaaaccagtgacctccggttgctttccgattatgcatgatcgcacgaaaatccgtcagctgccgaacctgctgcgtggctacgaaaacattcgtctgtccactcagaacgttatcgacgcagaaaaagcgccgggtggcccgtaccgtctgggtacctctggtagctgcccgaacgcaacgtccaaaagcggtttcttcgcaaccatggcatgggccgttccgaaggacaacaacaaaaacgctacgaatccactgactgttgaggtgccgtacatttgcaccgaaggcgaagaccaaatcaccgtatggggcttccactccgatgacaaaacccagatgaaaaacctgtacggtgactccaaccctcagaagttcacttcttctgcgaacggcgttacgacccactacgttagccaaattggttcctttccggaccagaccgaagacggcggtctgccgcagtctggtcgtatcgtggttgattatatgatgcagaaaccgggcaaaaccggcacgatcgtttatcagcgtggtgtgctgctgccgcagaaggtttggtgcgcgtctggtcgctccaaagtaatcaaaggttcagttgtttcagcagatgctaaaaatgccttaattgctggcggcgttgacgctaccgatgctaatggcgctgagttggtcaaaatgtcttataccgataaaaatggtaagacaattgaaggcggttatgcgcttaaagctggcgataagtattacgccgcagattacgatgaagcgacaggagcaattaaagctaaaaccacaagttatactgctgctgacggcactaccaaaacagcggctaaccaactgggtggcgtagacggtaaaaccgaagtcgttactatcgacggtaaaacctacaatgccagcaaagccgctggtcatgatttcaaagcacaaccagagctggcggaagcagccgctaaaaccaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattttgcagcaggccggtacttccgttctggcgcaggctaaccaggtcccgcagaacgtgctgagcctgttagcgggtactcgtacccgcggtaaactgtgcccagattgcctgaactgcaccgacctggacgtagcactgggccgtcctatgtgcgtgggtactacgccgtctgcaaaagctagcattctgcacgaggttaaaccagtgacctccggttgctttccgattatgcatgatcgcacgaaaatccgtcagctgccgaacctgctgcgtggctacgaaaacattcgtctgtccactcagaacgttatcgacgcagaaaaagcgccgggtggcccgtaccgtctgggtacctctggtagctgcccgaacgcaacgtccaaaagcggtttcttcgcaaccatggcatgggccgttccgaaggacaacaacaaaaacgctacgaatccactgactgttgaggtgccgtacatttgcaccgaaggcgaagaccaaatcaccgtatggggcttccactccgatgacaaaacccagatgaaaaacctgtacggtgactccaaccctcagaagttcacttcttctgcgaacggcgttacgacccactacgttagccaaattggttcctttccggaccagaccgaagacggcggtctgccgcagtctggtcgtatcgtggttgattatatgatgcagaaaccgggcaaaaccggcacgatcgtttatcagcgtggtgtgctgctgccgcagaaggtttggtgcgcgtctggtcgctccaaagtaatcaaaggttagtaggctgagc

SEQ ID NO: 2 - protein, 883 aa, Artificial Sequence
  HL118 R3-2xHA1-2 FL
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKASTRTRGKLCPDCLNCTDLDVALGRPMCVGTTPSAKASILHEVKPVTSGCFPIMHDRTKIRQLPNLLRGYENIRLSTQNVIDAEKAPGGPYRLGTSGSCPNATSKSGFFATMAWAVPKDNNKNATNPLTVEVPYICTEGEDQITVWGFHSDDKTQMKNLYGDSNPQKFTSSANGVTTHYVSQIGSFPDQTEDGGLPQSGRIVVDYMMQKPGKTGTIVYQRGVLLPQKVWCASGRSKVIKGSVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLAGTRTRGKLCPDCLNCTDLDVALGRPMCVGTTPSAKASILHEVKPVTSGCFPIMHDRTKIRQLPNLLRGYENIRLSTQNVIDAEKAPGGPYRLGTSGSCPNATSKSGFFATMAWAVPKDNNKNATNPLTVEVPYICTEGEDQITVWGFHSDDKTQMKNLYGDSNPQKFTSSANGVTTHYVSQIGSFPDQTEDGGLPQSGRIVVDYMMQKPGKTGTIVYQRGVLLPQKVWCASGRSKVIKG

SEQ ID NO: 3 - DNA, 2663 bp, Artificial Sequence
  HL163 STF2R3.2xHA1-2 FL (WT)
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagaaagcgtatgatgtgaaagatacagcagtaacaacgaaagctggaacaaggaccagagggaaactatgcccagactgtctcaactgcacagatctggatgtggctttgggcagaccaatgtgtgtggggaccacaccttcggcgaaagcttcaatactccacgaagtcaaacctgttacatccgggtgctttcctataatgcacgacagaacaaaaatcaggcaactacccaatcttctcagaggatatgaaaatatcaggctatcaacccaaaacgtcatcgatgcggaaaaggcaccaggaggaccctacagacttggaacctcaggatcttgccctaacgctaccagtaagagcggatttttcgcaacaatggcttgggctgtcccaaaggacaacaacaaaaatgcaacgaacccactaacagtagaagtaccatacatttgtacagaaggggaagaccaaatcactgtttgggggttccattcagatgacaaaacccaaatgaagaacctctatggagactcaaatcctcaaaagttcacctcatctgctaatggagtaaccacacactatgtttctcagattggcagcttcccagatcaaacagaagacggaggactaccacaaagcggcaggattgttgttgattacatgatgcaaaaacctgggaaaacaggaactattgtctaccaaagaggtgttttgttgcctcaaaaggtgtggtgcgcgagtggcaggagcaaagtaataaaagggtcagttgtttcagcagatgctaaaaatgccttaattgctggcggcgttgacgctaccgatgctaatggcgctgagttggtcaaaatgtcttataccgataaaaatggtaagacaattgaaggcggttatgcgcttaaagctggcgataagtattacgccgcagattacgatgaagcgacaggagcaattaaagctaaaactacaagttatactgctgctgacggcactaccaaaacagcggctaaccaactgggtggcgtagacggtaaaaccgaagtcgttactatcgacggtaaaacctacaatgccagcaaagccgctggtcatgatttcaaagcacaaccagagctggcggaagcagccgctaaaaccaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattctgcagcaggccggtacttccgttctggcgcaggctaaccaggtcccgcagaacgtgctgtctctgttagcgggaacaaggaccagagggaaactatgcccagactgtctcaactgcacagatctggatgtggctttgggcagaccaatgtgtgtggggaccacaccttcggcgaaagcttcaatactccacgaagtcaaacctgttacatccgggtgctttcctataatgcacgacagaacaaaaatcaggcaactacccaatcttctcagaggatatgaaaatatcaggctatcaacccaaaacgtcatcgatgcggaaaaggcaccaggaggaccctacagacttggaacctcaggatcttgccctaacgctaccagtaagagcggatttttcgcaacaatggcttgggctgtcccaaaggacaacaacaaaaatgcaacgaacccactaacagtagaagtaccatacatttgtacagaaggggaagaccaaatcactgtttgggggttccattcagatgacaaaacccaaatgaagaacctctatggagactcaaatcctcaaaagttcacctcatctgctaatggagtaaccacacactatgtttctcagattggcagcttcccagatcaaacagaagacggaggactaccacaaagcggcaggattgttgttgattacatgatgcaaaaacctgggaaaacaggaacaattgtctaccaaagaggtgttttgttgcctcaaaaggtgtggtgcgcgagtggcaggagcaaagtaataaaagggtagaattcgagctc

SEQ ID NO: 4 - protein, 883 aa, Artificial Sequence
  HL163 STF2R3.2xHA1-2 FL (WT)
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAGTRTRGKLCPDCLNCTDLDVALGRPMCVGTTPSAKASILHEVKPVTSGCFPIMHDRTKIRQLPNLLRGYENIRLSTQNVIDAEKAPGGPYRLGTSGSCPNATSKSGFFATMAWAVPKDNNKNATNPLTVEVPYICTEGEDQITVWGFHSDDKTQMKNLYGDSNPQKFTSSANGVTTHYVSQIGSFPDQTEDGGLPQSGRIVVDYMMQKPGKTGTIVYQRGVLLPQKVWCASGRSKVIKGSVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLAGTRTRGKLCPDCLNCTDLDVALGRPMCVGTTPSAKASILHEVKPVTSGCFPIMHDRTKIRQLPNLLRGYENIRLSTQNVIDAEKAPGGPYRLGTSGSCPNATSKSGFFATMAWAVPKDNNKNATNPLTVEVPYICTEGEDQITVWGFHSDDKTQMKNLYGDSNPQKFTSSANGVTTHYVSQIGSFPDQTEDGGLPQSGRIVVDYMMQKPGKTGTIVYQRGVLLPQKVWCASGRSKVIKG

SEQ ID NO: 5 - DNA, 1538 bp, Artificial Sequence
  HL199 STF2R23.HA1-2 FL (virus WT seq)
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagggaacaaggaccagagggaaactatgcccagactgtctcaactgcacagatctggatgtggctttgggcagaccaatgtgtgtggggaccacaccttcggcgaaagcttcaatactccacgaagtcaaacctgttacatccgggtgctttcctataatgcacgacagaacaaaaatcaggcaactacccaatcttctcagaggatatgaaaatatcaggctatcaacccaaaacgtcatcgatgcggaaaaggcaccaggaggaccctacagacttggaacctcaggatcttgccctaacgctaccagtaagagcggatttttcgcaacaatggcttgggctgtcccaaaggacaacaacaaaaatgcaacgaacccactaacagtagaagtaccatacatttgtacagaaggggaagaccaaatcactgtttgggggttccattcagatgacaaaacccaaatgaagaacctctatggagactcaaatcctcaaaagttcacctcatctgctaatggagtaaccacacactatgtttctcagattggcagcttcccagatcaaacagaagacggaggactaccacaaagcggcaggattgttgttgattacatgatgcaaaaacctgggaaaacaggaactattgtctaccaaagaggtgttttgttgcctcaaaaggtgtggtgcgcgagtggcaggagcaaagtaataaaagggaaaaccaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattctgcagcaggccggtacttccgttctggcgcaggctaaccaggtcccgcagaacgtgctgagcctgttacgttagaattcgagctc

SEQ ID NO: 6 - protein, 508 aa, Artificial Sequence
  HL199 STF2R23.HA1-2 FL (virus WT seq)
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQGTRTRGKLCPDCLNCTDLDVALGRPMCVGTTPSAKASILHEVKPVTSGCFPIMHDRTKIRQLPNLLRGYENIRLSTQNVIDAEKAPGGPYRLGTSGSCPNATSKSGFFATMAWAVPKDNNKNATNPLTVEVPYICTEGEDQITVWGFHSDDKTQMKNLYGDSNPQKFTSSANGVTTHYVSQIGSFPDQTEDGGLPQSGRIVVDYMMQKPGKTGTIVYQRGVLLPQKVWCASGRSKVIKGKTTENPLQKIDAALAQVDALRSDLGAVQNRFNSAITNLGNTVNNLSEARSRIEDSDYATEVSNMSRAQILQQAGTSVLAQANQVPQNVLSLLR

SEQ ID NO: 7 - DNA, 1556 bp, Artificial Sequence
  HL261 STF2R23.HA1-2 FL-GSG linkers (WT seq; with GSG linkers between STF2 and HA1-2)
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagggatctggtggaacaaggaccagagggaaactatgcccagactgtctcaactgcacagatctggatgtggctttgggcagaccaatgtgtgtggggaccacaccttcggcgaaagcttcaatactccacgaagtcaaacctgttacatccgggtgctttcctataatgcacgacagaacaaaaatcaggcaactacccaatcttctcagaggatatgaaaatatcaggctatcaacccaaaacgtcatcgatgcggaaaaggcaccaggaggaccctacagacttggaacctcaggatcttgccctaacgctaccagtaagagcggatttttcgcaacaatggcttgggctgtcccaaaggacaacaacaaaaatgcaacgaacccactaacagtagaagtaccatacatttgtacagaaggggaagaccaaatcactgtttgggggttccattcagatgacaaaacccaaatgaagaacctctatggagactcaaatcctcaaaagttcacctcatctgctaatggagtaaccacacactatgtttctcagattggcagcttcccagatcaaacagaagacggaggactaccacaaagcggcaggattgttgttgattacatgatgcaaaaacctgggaaaacaggaactattgtctaccaaagaggtgttttgttgcctcaaaaggtgtggtgcgcgagtggcaggagcaaagtaataaaagggggtagcggaaaaaccaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattctgcagcaggccggtacttccgttctggcgcaggctaaccaggtcccgcagaacgtgctgagcctgttacgttagaattcgagctc

SEQ ID NO: 8 - protein, 514 aa, Artificial Sequence
  HL261 STF2R23.HA1-2 FL-GSG linkers (WT seq; with GSG linkers between STF2 and HA1-2)
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQGSGGTRTRGKLCPDCLNCTDLDVALGRPMCVGTTPSAKASILHEVKPVTSGCFPIMHDRTKIRQLPNLLRGYENIRLSTQNVIDAEKAPGGPYRLGTSGSCPNATSKSGFFATMAWAVPKDNNKNATNPLTVEVPYICTEGEDQITVWGFHSDDKTQMKNLYGDSNPQKFTSSANGVTTHYVSQIGSFPDQTEDGGLPQSGRIVVDYMMQKPGKTGTIVYQRGVLLPQKVWCASGRSKVIKGGSGKTTENPLQKIDAALAQVDALRSDLGAVQNRFNSAITNLGNTVNNLSEARSRIEDSDYATEVSNMSRAQILQQAGTSVLAQANQVPQNVLSLLR

SEQ ID NO: 9 - DNA, 1952 bp, Artificial Sequence
  HL351 STF2R3 HA1-2 FL-1-Loop replace D3 seq (cLoop=12aa replaced 10aa of D3)
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagaaagcgtatgatgtgaaagatacagcagtaacaacgaaagctggaacaaggaccagagggaaactatgcccagactgtctcaactgcacagatctggatgtggctttgggcagaccaatgtgtgtggggaccacaccttcggcgaaagcttcaatactccacgaagtcaaacctgttacatccgggtgctttcctataatgcacgacagaacaaaaatcaggcaactacccaatcttctcagaggatatgaaaatatcaggctatcaacccaaaacgtcatcgatgcggaaaaggcaccaggaggaccctacagacttggaacctcaggatcttgccctaacgctaccagtaagagcggatttttcgcaacaatggcttgggctgtcccaaaggacaacaacaaaaatgcaacgaacccactaacagtagaagtaccatacatttgtacagaaggggaagaccaaatcactgtttgggggttccattcagatgacaaaacccaaatgaagaacctctatggagactcaaatcctcaaaagttcacctcatctgctaatggagtaaccacacactatgtttctcagattggcagcttcccagatcaaacagaagacggaggactaccacaaagcggcaggattgttgttgattacatgatgcaaaaacctgggaaaacaggaacaattgtctaccaaagaggtgttttgttgcctcaaaaggtgtggtgcgcgagtggcaggagcaaagtaataaaagggtcactggtggttagcaccttcaaagcgagcgccacgggtattgctggcggcgttgacgctaccgatgctaatggcgctgagttggtcaaaatgtcttataccgataaaaatggtaagacaattgaaggcggttatgcgcttaaagctggcgataagtattacgccgcagattacgatgaagcgacaggagcaattaaagctaaaaccacaagttatactgctgctgacggcactaccaaaacagcggctaaccaactgggtggcgtagacggtaaaaccgaagtcgttactatcgacggtaaaacctacaatgccagcaaagccgctggtcatgatttcaaagcacaaccagagctggcggaagcagccgctaaaaccaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattttgcagcaggccggtacttccgttctggcgcaggctaaccaggtcccgcagaacgtgctgagcctgttacgttagaattcgagctc

SEQ ID NO: 10 - protein, 646 aa, Artificial Sequence
  HL351 STF2R3 HA1-2 FL-1-Loop replace D3 seq (cLoop=12aa replaced 10aa of D3)
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAGTRTRGKLCPDCLNCTDLDVALGRPMCVGTTPSAKASILHEVKPVTSGCFPIMHDRTKIRQLPNLLRGYENIRLSTQNVIDAEKAPGGPYRLGTSGSCPNATSKSGFFATMAWAVPKDNNKNATNPLTVEVPYICTEGEDQITVWGFHSDDKTQMKNLYGDSNPQKFTSSANGVTTHYVSQIGSFPDQTEDGGLPQSGRIVVDYMMQKPGKTGTIVYQRGVLLPQKVWCASGRSKVIKGSLVVSTFKASATGIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLR

SEQ ID NO: 11 - DNA, 1970 bp, Artificial Sequence
  HL352 STF2R3 HA1-2 FL-2-Elongation of C-term (CT; add 9aa in C-term of HA1-2)
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagaaagcgtatgatgtgaaagatacagcagtaacaacgaaagctggaacaaggaccagagggaaactatgcccagactgtctcaactgcacagatctggatgtggctttgggcagaccaatgtgtgtggggaccacaccttcggcgaaagcttcaatactccacgaagtcaaacctgttacatccgggtgctttcctataatgcacgacagaacaaaaatcaggcaactacccaatcttctcagaggatatgaaaatatcaggctatcaacccaaaacgtcatcgatgcggaaaaggcaccaggaggaccctacagacttggaacctcaggatcttgccctaacgctaccagtaagagcggatttttcgcaacaatggcttgggctgtcccaaaggacaacaacaaaaatgcaacgaacccactaacagtagaagtaccatacatttgtacagaaggggaagaccaaatcactgtttgggggttccattcagatgacaaaacccaaatgaagaacctctatggagactcaaatcctcaaaagttcacctcatctgctaatggagtaaccacacactatgtttctcagattggcagcttcccagatcaaacagaagacggaggactaccacaaagcggcaggattgttgttgattacatgatgcaaaaacctgggaaaacaggaacaattgtctaccaaagaggtgttttgttgcctcaaaaggtgtggtgcgcgagtggcaggagcaaagtaataaaagggtcactgccgttgatcggtgaagcggatgttgtttcagcagatgctaaaaatgccttaattgctggcggcgttgacgctaccgatgctaatggcgctgagttggtcaaaatgtcttataccgataaaaatggtaagacaattgaaggcggttatgcgcttaaagctggcgataagtattacgccgcagattacgatgaagcgacaggagcaattaaagctaaaaccacaagttatactgctgctgacggcactaccaaaacagcggctaaccaactgggtggcgtagacggtaaaaccgaagtcgttactatcgacggtaaaacctacaatgccagcaaagccgctggtcatgatttcaaagcacaaccagagctggcggaagcagccgctaaaaccaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattttgcagcaggccggtacttccgttctggcgcaggctaaccaggtcccgcagaacgtgctgagcctgttacgttagaattcgagctc

SEQ ID NO: 12 - protein, 652 aa, Artificial Sequence
  HL352 STF2R3 HA1-2 FL-2-Elongation of C-term (CT; add 9aa in C-term of HA1-2)
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAGTRTRGKLCPDCLNCTDLDVALGRPMCVGTTPSAKASILHEVKPVTSGCFPIMHDRTKIRQLPNLLRGYENIRLSTQNVIDAEKAPGGPYRLGTSGSCPNATSKSGFFATMAWAVPKDNNKNATNPLTVEVPYICTEGEDQITVWGFHSDDKTQMKNLYGDSNPQKFTSSANGVTTHYVSQIGSFPDQTEDGGLPQSGRIVVDYMMQKPGKTGTIVYQRGVLLPQKVWCASGRSKVIKGSLPLIGEADVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLR

SEQ ID NO: 13 - DNA, 1970 bp, Artificial Sequence
  HL354 STF2R3 HA1-2 FL-4-(nGGG+cLGGG+cLoop)
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagaaagcgtatgatgtgaaagatacagcagtaacaacgaaagctggcggtggcggaacaaggaccagagggaaactatgcccagactgtctcaactgcacagatctggatgtggctttgggcagaccaatgtgtgtggggaccacaccttcggcgaaagcttcaatactccacgaagtcaaacctgttacatccgggtgctttcctataatgcacgacagaacaaaaatcaggcaactacccaatcttctcagaggatatgaaaatatcaggctatcaacccaaaacgtcatcgatgcggaaaaggcaccaggaggaccctacagacttggaacctcaggatcttgccctaacgctaccagtaagagcggatttttcgcaacaatggcttgggctgtcccaaaggacaacaacaaaaatgcaacgaacccactaacagtagaagtaccatacatttgtacagaaggggaagaccaaatcactgtttgggggttccattcagatgacaaaacccaaatgaagaacctctatggagactcaaatcctcaaaagttcacctcatctgctaatggagtaaccacacactatgtttctcagattggcagcttcccagatcaaacagaagacggaggactaccacaaagcggcaggattgttgttgattacatgatgcaaaaacctgggaaaacaggaacaattgtctaccaaagaggtgttttgttgcctcaaaaggtgtggtgcgcgagtggcaggagcaaagtaataaaagggtcactgggtggcggtgtggttagcaccttcaaagcgagcgccacgggtttggctggcggcgttgacgctaccgatgctaatggcgctgagttggtcaaaatgtcttataccgataaaaatggtaagacaattgaaggcggttatgcgcttaaagctggcgataagtattacgccgcagattacgatgaagcgacaggagcaattaaagctaaaaccacaagttatactgctgctgacggcactaccaaaacagcggctaaccaactgggtggcgtagacggtaaaaccgaagtcgttactatcgacggtaaaacctacaatgccagcaaagccgctggtcatgatttcaaagcacaaccagagctggcggaagcagccgctaaaaccaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattttgcagcaggccggtacttccgttctggcgcaggctaaccaggtcccgcagaacgtgctgagcctgttacgttagaattcgagctc

SEQ ID NO: 14 - protein, 652 aa, Artificial Sequence
  HL354 STF2R3 HA1-2 FL-4-(nGGG+cLGGG+cLoop)
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAGGGGTRTRGKLCPDCLNCTDLDVALGRPMCVGTTPSAKASILHEVKPVTSGCFPIMHDRTKIRQLPNLLRGYENIRLSTQNVIDAEKAPGGPYRLGTSGSCPNATSKSGFFATMAWAVPKDNNKNATNPLTVEVPYICTEGEDQITVWGFHSDDKTQMKNLYGDSNPQKFTSSANGVTTHYVSQIGSFPDQTEDGGLPQSGRIVVDYMMQKPGKTGTIVYQRGVLLPQKVWCASGRSKVIKGSLGGGVVSTFKASATGLAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLR

SEQ ID NO: 15 - DNA, 1967 bp, Artificial Sequence
  HL357 STF2R3 HA1-2 FL-7-(nGGG+cLGGG)
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagaaagcgtatgatgtgaaagatacagcagtaacaacgaaagctggcggtggcggaacaaggaccagagggaaactatgcccagactgtctcaactgcacagatctggatgtggctttgggcagaccaatgtgtgtggggaccacaccttcggcgaaagcttcaatactccacgaagtcaaacctgttacatccgggtgctttcctataatgcacgacagaacaaaaatcaggcaactacccaatcttctcagaggatatgaaaatatcaggctatcaacccaaaacgtcatcgatgcggaaaaggcaccaggaggaccctacagacttggaacctcaggatcttgccctaacgctaccagtaagagcggatttttcgcaacaatggcttgggctgtcccaaaggacaacaacaaaaatgcaacgaacccactaacagtagaagtaccatacatttgtacagaaggggaagaccaaatcactgtttgggggttccattcagatgacaaaacccaaatgaagaacctctatggagactcaaatcctcaaaagttcacctcatctgctaatggagtaaccacacactatgtttctcagattggcagcttcccagatcaaacagaagacggaggactaccacaaagcggcaggattgttgttgattacatgatgcaaaaacctgggaaaacaggaacaattgtctaccaaagaggtgttttgttgcctcaaaaggtgtggtgcgcgagtggcaggagcaaagtaataaaagggtcacttggtggcggtgttgtttcagcagatgctaaaaatgccttaattgctggcggcgttgacgctaccgatgctaatggcgctgagttggtcaaaatgtcttataccgataaaaatggtaagacaattgaaggcggttatgcgcttaaagctggcgataagtattacgccgcagattacgatgaagcgacaggagcaattaaagctaaaaccacaagttatactgctgctgacggcactaccaaaacagcggctaaccaactgggtggcgtagacggtaaaaccgaagtcgttactatcgacggtaaaacctacaatgccagcaaagccgctggtcatgatttcaaagcacaaccagagctggcggaagcagccgctaaaaccaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattttgcagcaggccggtacttccgttctggcgcaggctaaccaggtcccgcagaacgtgctgagcctgttacgttagaattcgagctc

SEQ ID NO: 16 - protein, 651 aa, Artificial Sequence
  HL357 STF2R3 HA1-2 FL-7-(nGGG+cLGGG)
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAGGGGTRTRGKLCPDCLNCTDLDVALGRPMCVGTTPSAKASILHEVKPVTSGCFPIMHDRTKIRQLPNLLRGYENIRLSTQNVIDAEKAPGGPYRLGTSGSCPNATSKSGFFATMAWAVPKDNNKNATNPLTVEVPYICTEGEDQITVWGFHSDDKTQMKNLYGDSNPQKFTSSANGVTTHYVSQIGSFPDQTEDGGLPQSGRIVVDYMMQKPGKTGTIVYQRGVLLPQKVWCASGRSKVIKGSLGGGVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLR

SEQ ID NO: 17 - DNA, 1944 bp, Artificial Sequence
  HL384 STF2R3.HA1-2 Sich (B/99)-Corrected
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagaaagcgtatgatgtgaaagatacagcagtaacaacgaaagctaaaggtacgaaaacccgtggcaaactgtgtccgacgtgccttaactgcacggacctcgatgtagcgctgggtcgcccgatgtgcgtcggaattaccccgtcggcgaaggcaagtattttacatgaaatcaaacctgtcacatccggctgctttccgattatgcatgatcgtacgaaaatccgccagcttccgaacctgctgcgcggttacgaaaaaatccgtctttcgacacaaaacgtcatcaacgcggaaaaagcaccgggcggcccgtatcgtctgggcacaagcggctcttgtccgaacgccacgagcaaatcaggtttcttcgcgaccatggcctgggcggttcctcgcgataataataaaaccgccaccaaccctctgaccgttgaagtccctcatatctgcaccaaagaagaggatcagatcactgtttgggggtttcattcggatgataaaactcagatgaaaaacctgtacggtgattccaatccccagaaattcacaagcagcgccaacggtatcaccacccattacgtgagtcagattggtggttttccggaccaaaccgaagatggcgggctcccacagagtggccgtattgttgtggattatatggtacaaaaaccgggtaaaaccggcaccatcgtttatcagcgcgggatcctgctgccacaaaaagtctggtgcgcgagcggccgttcgaaagtcattaaaggtgcagttgtttcagcagatgctaaaaatgccttaattgctggcggcgttgacgctaccgatgctaatggcgctgagttggtcaaaatgtcttataccgataaaaatggtaagacaattgaaggcggttatgcgcttaaagctggcgataagtattacgccgcagattacgatgaagcgacaggagcaattaaagctaaaaccacaagttatactgctgctgacggcactaccaaaacagcggctaaccaactgggtggcgtagacggtaaaaccgaagtcgttactatcgacggtaaaacctacaatgccagcaaagccgctggtcatgatttcaaagcacaaccagagctggcggaagcagccgctaaaaccaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattttgcagcaggccggtacttccgttctggcgcaggctaaccaggtcccgcagaacgtgctgagcctgttacgttagctcgag

SEQ ID NO: 18 - protein, 645 aa, Artificial Sequence
  HL384 STF2R3.HA1-2 Sich (B/99)-Corrected
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAKGTKTRGKLCPTCLNCTDLDVALGRPMCVGITPSAKASILHEIKPVTSGCFPIMHDRTKIRQLPNLLRGYEKIRLSTQNVINAEKAPGGPYRLGTSGSCPNATSKSGFFATMAWAVPRDNNKTATNPLTVEVPHICTKEEDQITVWGFHSDDKTQMKNLYGDSNPQKFTSSANGITTHYVSQIGGFPDQTEDGGLPQSGRIVVDYMVQKPGKTGTIVYQRGILLPQKVWCASGRSKVIKGAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLR

SEQ ID NO: 19 - DNA, 1529 bp, Artificial Sequence
  HL389 STF2R23.HA1-2 FLs (further remove 3aa from C-term half of flagellin)
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagggaacaaggaccagagggaaactatgcccagactgtctcaactgcacagatctggatgtggctttgggcagaccaatgtgtgtggggaccacaccttcggcgaaagcttcaatactccacgaagtcaaacctgttacatccgggtgctttcctataatgcacgacagaacaaaaatcaggcaactacccaatcttctcagaggatatgaaaatatcaggctatcaacccaaaacgtcatcgatgcggaaaaggcaccaggaggaccctacagacttggaacctcaggatcttgccctaacgctaccagtaagagcggatttttcgcaacaatggcttgggctgtcccaaaggacaacaacaaaaatgcaacgaacccactaacagtagaagtaccatacatttgtacagaaggggaagaccaaatcactgtttgggggttccattcagatgacaaaacccaaatgaagaacctctatggagactcaaatcctcaaaagttcacctcatctgctaatggagtaaccacacactatgtttctcagattggcagcttcccagatcaaacagaagacggaggactaccacaaagcggcaggattgttgttgattacatgatgcaaaaacctgggaaaacaggaactattgtctaccaaagaggtgttttgttgcctcaaaaggtgtggtgcgcgagtggcaggagcaaagtaataaaaggggaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattctgcagcaggccggtacttccgttctggcgcaggctaaccaggtcccgcagaacgtgctgagcctgttacgttagaattcgagctc

SEQ ID NO: 20 - protein, 505 aa, Artificial Sequence
  HL389 STF2R23.HA1-2 FLs (further remove 3aa from C-term half of flagellin)
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQGTRTRGKLCPDCLNCTDLDVALGRPMCVGTTPSAKASILHEVKPVTSGCFPIMHDRTKIRQLPNLLRGYENIRLSTQNVIDAEKAPGGPYRLGTSGSCPNATSKSGFFATMAWAVPKDNNKNATNPLTVEVPYICTEGEDQITVWGFHSDDKTQMKNLYGDSNPQKFTSSANGVTTHYVSQIGSFPDQTEDGGLPQSGRIVVDYMMQKPGKTGTIVYQRGVLLPQKVWCASGRSKVIKGENPLQKIDAALAQVDALRSDLGAVQNRFNSAITNLGNTVNNLSEARSRIEDSDYATEVSNMSRAQILQQAGTSVLAQANQVPQNVLSLLA

SEQ ID NO: 21 - DNA, 1565 bp, Artificial Sequence
  HL406 STF2R23 HA1-2 FL (Extend 9aa in C-term of HA1-2)
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagggaacaaggaccagagggaaactatgcccagactgtctcaactgcacagatctggatgtggctttgggcagaccaatgtgtgtggggaccacaccttcggcgaaagcttcaatactccacgaagtcaaacctgttacatccgggtgctttcctataatgcacgacagaacaaaaatcaggcaactacccaatcttctcagaggatatgaaaatatcaggctatcaacccaaaacgtcatcgatgcggaaaaggcaccaggaggaccctacagacttggaacctcaggatcttgccctaacgctaccagtaagagcggatttttcgcaacaatggcttgggctgtcccaaaggacaacaacaaaaatgcaacgaacccactaacagtagaagtaccatacatttgtacagaaggggaagaccaaatcactgtttgggggttccattcagatgacaaaacccaaatgaagaacctctatggagactcaaatcctcaaaagttcacctcatctgctaatggagtaaccacacactatgtttctcagattggcagcttcccagatcaaacagaagacggaggactaccacaaagcggcaggattgttgttgattacatgatgcaaaaacctgggaaaacaggaactattgtctaccaaagaggtgttttgttgcctcaaaaggtgtggtgcgcgagtggcaggagcaaagtaataaaagggagcctgccgctgattggcgaagcggataaaaccaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattctgcagcaggccggtacttccgttctggcgcaggctaaccaggtcccgcagaacgtgctgagcctgttacgttagaattcgagctc

SEQ ID NO: 22 - protein, 517 aa, Artificial Sequence
  HL406 STF2R23 HA1-2 FL (Extend 9aa in C-term of HA1-2)
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQGTRTRGKLCPDCLNCTDLDVALGRPMCVGTTPSAKASILHEVKPVTSGCFPIMHDRTKIRQLPNLLRGYENIRLSTQNVIDAEKAPGGPYRLGTSGSCPNATSKSGFFATMAWAVPKDNNKNATNPLTVEVPYICTEGEDQITVWGFHSDDKTQMKNLYGDSNPQKFTSSANGVTTHYVSQIGSFPDQTEDGGLPQSGRIVVDYMMQKPGKTGTIVYQRGVLLPQKVWCASGRSKVIKGSLPLIGEADKTTENPLQKIDAALAQVDALRSDLGAVQNRFNSAITNLGNTVNNLSEARSRIEDSDYATEVSNMSRAQILQQAGTSVLAQANQVPQNVLSLLR

SEQ ID NO: 23 - DNA, 1970 bp, Artificial Sequence
  HL487 G to S mutation of HL352 (STF2R3 HA1-2 FL-9 aa elongation of C-term)
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagaaagcgtatgatgtgaaagatacagcagtaacaacgaaagctagcacaaggaccagagggaaactatgcccagactgtctcaactgcacagatctggatgtggctttgggcagaccaatgtgtgtggggaccacaccttcggcgaaagcttcaatactccacgaagtcaaacctgttacatccgggtgctttcctataatgcacgacagaacaaaaatcaggcaactacccaatcttctcagaggatatgaaaatatcaggctatcaacccaaaacgtcatcgatgcggaaaaggcaccaggaggaccctacagacttggaacctcaggatcttgccctaacgctaccagtaagagcggatttttcgcaacaatggcttgggctgtcccaaaggacaacaacaaaaatgcaacgaacccactaacagtagaagtaccatacatttgtacagaaggggaagaccaaatcactgtttgggggttccattcagatgacaaaacccaaatgaagaacctctatggagactcaaatcctcaaaagttcacctcatctgctaatggagtaaccacacactatgtttctcagattggcagcttcccagatcaaacagaagacggaggactaccacaaagcggcaggattgttgttgattacatgatgcaaaaacctgggaaaacaggaacaattgtctaccaaagaggtgttttgttgcctcaaaaggtgtggtgcgcgagtggcaggagcaaagtaataaaagggtcactgccgttgatcggtgaagcggatgttgtttcagcagatgctaaaaatgccttaattgctggcggcgttgacgctaccgatgctaatggcgctgagttggtcaaaatgtcttataccgataaaaatggtaagacaattgaaggcggttatgcgcttaaagctggcgataagtattacgccgcagattacgatgaagcgacaggagcaattaaagctaaaaccacaagttatactgctgctgacggcactaccaaaacagcggctaaccaactgggtggcgtagacggtaaaaccgaagtcgttactatcgacggtaaaacctacaatgccagcaaagccgctggtcatgatttcaaagcacaaccagagctggcggaagcagccgctaaaaccaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattttgcagcaggccggtacttccgttctggcgcaggctaaccaggtcccgcagaacgtgctgagcctgttacgttagaattcgagctc

SEQ ID NO: 24 - protein, 652 aa, Artificial Sequence
  HL487 G to S mutation of HL352 (STF2R3 HA1-2 FL-9 aa elongation of C-term)
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKASTRTRGKLCPDCLNCTDLDVALGRPMCVGTTPSAKASILHEVKPVTSGCFPIMHDRTKIRQLPNLLRGYENIRLSTQNVIDAEKAPGGPYRLGTSGSCPNATSKSGFFATMAWAVPKDNNKNATNPLTVEVPYICTEGEDQITVWGFHSDDKTQMKNLYGDSNPQKFTSSANGVTTHYVSQIGSFPDQTEDGGLPQSGRIVVDYMMQKPGKTGTIVYQRGVLLPQKVWCASGRSKVIKGSLPLIGEADVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLR

SEQ ID NO: 25 - DNA, 2511 bp, Artificial Sequence
  HL399 STF2.HA1 Perth (Long HA-add 51aa in N term, 57aa in C-term of HA1-2)
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagaaagcgtatgatgtgaaagatacagcagtaacaacgaaagcttatgccaataatggtactacactggatgtatcgggtcttgatgatgcagctattaaagcggctacgggtggtacgaatggtacggcttctgtaaccggtggtgcggttaaatttgacgcagataataacaagtactttgttactattggtggctttactggtgctgatgccgccaaaaatggcgattatgaagttaacgttgctactgacggtacagtaacccttgcggctggcgcaactaaaaccacaatgcctgctggtgcgacaactaaaacagaagtacaggagttaaaagatacaccggcagttgtttcagcagatgctaaaaatgccttaattgctggcggcgttgacgctaccgatgctaatggcgctgagttggtcaaaatgtcttataccgataaaaatggtaagacaattgaaggcggttatgcgcttaaagctggcgataagtattacgccgcagattacgatgaagcgacaggagcaattaaagctaaaactacaagttatactgctgctgacggcactaccaaaacagcggctaaccaactgggtggcgtagacggtaaaaccgaagtcgttactatcgacggtaaaacctacaatgccagcaaagccgctggtcatgatttcaaagcacaaccagagctggcggaagcagccgctaaaaccaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattctgcagcaggccggtacttccgttctggcgcaggctaaccaggtcccgcagaacgtgctgtctctgttagcgcaaaagttgcccggcaatgataacagtactgctactttgtgtttgggtcatcatgcggttcctaatggtactatcgtcaaaacaatcaccaacgaccagattgaggtaactaacgctaccgagctggtacaatcctcctctacgggcgaaatctgcgacagcccgcatcagattctggatggcaaaaactgcaccctgattgatgcgctgctgggcgatccgcagtgcgatggctttcagaacaaaaaatgggatctgtttgtggaacgcagcaaagcgtatagcaactgctatccgtatgatgtgccggattatgcgagcctgcgcagcctggtggcgagcagcggcaccctggaatttaacaacgaaagctttaactggaccggcgtgacccagaacggcaccagcagcgcgtgcattcgccgcagcaaaaacagcttttttagccgcctgaactggctgacccatctgaactttaaatatccggcgctgaacgtgaccatgccgaacaacgaacagtttgataaactgtatatttggggcgtgcatcatccgggcaccgataaagatcagatttttctgtatgcgcaggcgagcggccgcattaccgtgagcaccaaacgcagccagcagaccgtgagcccgaacattggcagccgcccgcgcgtgcgcaacattccgagccgcattagcatttattggaccattgtgaaaccgggcgatattctgctgattaacagcaccggcaacctgattgcgccgcgcggctattttaaaattcgcagcggcaaaagcagcattatgcgcagcgatgcaccgatcggcaagtgcaactcagaatgtattaccccgaatggttctatacctaacgacaaacctttccaaaacgtgaaccgcattacctacggtgcctgtcccaggtatgtcaagcaaaatacactgaaactcgctacgggcatgagaaatgtgcctgaaaagcagacctagctcgag

SEQ ID NO: 26 - protein, 834 aa, Artificial Sequence
  HL399 STF2.HA1 Perth (Long HA-add 51aa in N term, 57aa in C-term of HA1-2)
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAYANNGTTLDVSGLDDAAIKAATGGTNGTASVTGGAVKFDADNNKYFVTIGGFTGADAAKNGDYEVNVATDGTVTLAAGATKTTMPAGATTKTEVQELKDTPAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLAQKLPGNDNSTATLCLGHHAVPNGTIVKTITNDQIEVTNATELVQSSSTGEICDSPHQILDGKNCTLIDALLGDPQCDGFQNKKWDLFVERSKAYSNCYPYDVPDYASLRSLVASSGTLEFNNESFNWTGVTQNGTSSACIRRSKNSFFSRLNWLTHLNFKYPALNVTMPNNEQFDKLYIWGVHHPGTDKDQIFLYAQASGRITVSTKRSQQTVSPNIGSRPRVRNIPSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQT

SEQ ID NO: 27 - DNA, 2208 bp, Artificial Sequence
  HL400 STF2R3.HA1 Perth (Long HA-add 51aa in N term, 57aa in C-term of HA1-2)
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagaaagcgtatgatgtgaaagatacagcagtaacaacgaaagctcaaaagttgcccggcaatgataacagtactgctactttgtgtttgggtcatcatgcggttcctaatggtactatcgtcaaaacaatcaccaacgaccagattgaggtaactaacgctaccgagctggtacaatcctcctctacgggcgaaatctgcgacagcccgcatcagattctggatggcaaaaactgcaccctgattgatgcgctgctgggcgatccgcagtgcgatggctttcagaacaaaaaatgggatctgtttgtggaacgcagcaaagcgtatagcaactgctatccgtatgatgtgccggattatgcgagcctgcgcagcctggtggcgagcagcggcaccctggaatttaacaacgaaagctttaactggaccggcgtgacccagaacggcaccagcagcgcgtgcattcgccgcagcaaaaacagcttttttagccgcctgaactggctgacccatctgaactttaaatatccggcgctgaacgtgaccatgccgaacaacgaacagtttgataaactgtatatttggggcgtgcatcatccgggcaccgataaagatcagatttttctgtatgcgcaggcgagcggccgcattaccgtgagcaccaaacgcagccagcagaccgtgagcccgaacattggcagccgcccgcgcgtgcgcaacattccgagccgcattagcatttattggaccattgtgaaaccgggcgatattctgctgattaacagcaccggcaacctgattgcgccgcgcggctattttaaaattcgcagcggcaaaagcagcattatgcgcagcgatgcaccgatcggcaagtgcaactcagaatgtattaccccgaatggttctatacctaacgacaaacctttccaaaacgtgaaccgcattacctacggtgcctgtcccaggtatgtcaagcaaaatacactgaaactcgctacgggcatgagaaatgtgcctgaaaagcagaccgcagttgtttcagcagatgctaaaaatgccttaattgctggcggcgttgacgctaccgatgctaatggcgctgagttggtcaaaatgtcttataccgataaaaatggtaagacaattgaaggcggttatgcgcttaaagctggcgataagtattacgccgcagattacgatgaagcgacaggagcaattaaagctaaaaccacaagttatactgctgctgacggcactaccaaaacagcggctaaccaactgggtggcgtagacggtaaaaccgaagtcgttactatcgacggtaaaacctacaatgccagcaaagccgctggtcatgatttcaaagcacaaccagagctggcggaagcagccgctaaaaccaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattttgcagcaggccggtacttccgttctggcgcaggctaaccaggtcccgcagaacgtgctgagcctgttacgttagctcgag

SEQ ID NO: 28 - protein, 733 aa, Artificial Sequence
  HL400 STF2R3.HA1 Perth (Long HA-add 51aa in N term, 57aa in C-term of HA1-2)
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAQKLPGNDNSTATLCLGHHAVPNGTIVKTITNDQIEVTNATELVQSSSTGEICDSPHQILDGKNCTLIDALLGDPQCDGFQNKKWDLFVERSKAYSNCYPYDVPDYASLRSLVASSGTLEFNNESFNWTGVTQNGTSSACIRRSKNSFFSRLNWLTHLNFKYPALNVTMPNNEQFDKLYIWGVHHPGTDKDQIFLYAQASGRITVSTKRSQQTVSPNIGSRPRVRNIPSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLR

SEQ ID NO: 29 - DNA, 2445 bp, Artificial Sequence
  HL401 STF2.HA1s Perth (Short version of HL399-N-17aa; C-5aa)
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagaaagcgtatgatgtgaaagatacagcagtaacaacgaaagcttatgccaataatggtactacactggatgtatcgggtcttgatgatgcagctattaaagcggctacgggtggtacgaatggtacggcttctgtaaccggtggtgcggttaaatttgacgcagataataacaagtactttgttactattggtggctttactggtgctgatgccgccaaaaatggcgattatgaagttaacgttgctactgacggtacagtaacccttgcggctggcgcaactaaaaccacaatgcctgctggtgcgacaactaaaacagaagtacaggagttaaaagatacaccggcagttgtttcagcagatgctaaaaatgccttaattgctggcggcgttgacgctaccgatgctaatggcgctgagttggtcaaaatgtcttataccgataaaaatggtaagacaattgaaggcggttatgcgcttaaagctggcgataagtattacgccgcagattacgatgaagcgacaggagcaattaaagctaaaactacaagttatactgctgctgacggcactaccaaaacagcggctaaccaactgggtggcgtagacggtaaaaccgaagtcgttactatcgacggtaaaacctacaatgccagcaaagccgctggtcatgatttcaaagcacaaccagagctggcggaagcagccgctaaaaccaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattctgcagcaggccggtacttccgttctggcgcaggctaaccaggtcccgcagaacgtgctgtctctgttagcgcatgcggttcctaatggtactatcgtcaaaacaatcaccaacgaccagattgaggtaactaacgctaccgagctggtacaatcctcctctacgggcgaaatctgcgacagcccgcatcagattctggatggcaaaaactgcaccctgattgatgcgctgctgggcgatccgcagtgcgatggctttcagaacaaaaaatgggatctgtttgtggaacgcagcaaagcgtatagcaactgctatccgtatgatgtgccggattatgcgagcctgcgcagcctggtggcgagcagcggcaccctggaatttaacaacgaaagctttaactggaccggcgtgacccagaacggcaccagcagcgcgtgcattcgccgcagcaaaaacagcttttttagccgcctgaactggctgacccatctgaactttaaatatccggcgctgaacgtgaccatgccgaacaacgaacagtttgataaactgtatatttggggcgtgcatcatccgggcaccgataaagatcagatttttctgtatgcgcaggcgagcggccgcattaccgtgagcaccaaacgcagccagcagaccgtgagcccgaacattggcagccgcccgcgcgtgcgcaacattccgagccgcattagcatttattggaccattgtgaaaccgggcgatattctgctgattaacagcaccggcaacctgattgcgccgcgcggctattttaaaattcgcagcggcaaaagcagcattatgcgcagcgatgcaccgatcggcaagtgcaactcagaatgtattaccccgaatggttctatacctaacgacaaacctttccaaaacgtgaaccgcattacctacggtgcctgtcccaggtatgtcaagcaaaatacactgaaactcgctacgggcatgagaaatgtgtagctcgag

SEQ ID NO: 30 - protein, 812 aa, Artificial Sequence
  HL401 STF2.HA1s Perth (Short version of HL399-N-17aa; C-5aa)
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAYANNGTTLDVSGLDDAAIKAATGGTNGTASVTGGAVKFDADNNKYFVTIGGFTGADAAKNGDYEVNVATDGTVTLAAGATKTTMPAGATTKTEVQELKDTPAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLAHAVPNGTIVKTITNDQIEVTNATELVQSSSTGEICDSPHQILDGKNCTLIDALLGDPQCDGFQNKKWDLFVERSKAYSNCYPYDVPDYASLRSLVASSGTLEFNNESFNWTGVTQNGTSSACIRRSKNSFFSRLNWLTHLNFKYPALNVTMPNNEQFDKLYIWGVHHPGTDKDQIFLYAQASGRITVSTKRSQQTVSPNIGSRPRVRNIPSRISIYWTIVKPGDILLINSTGNLIAPRGYFKIRSGKSSIMRSDAPIGKCNSECITPNGSIPNDKPFQNVNRITYGACPRYVKQNTLKLATGMRNV

SEQ ID NO: 31 - DNA, 2142 bp, Artificial Sequence
  HL402 STF2R3.HA1s Perth (Short version of HL399-N-17aa; C-5aa)
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagaaagcgtatgatgtgaaagatacagcagtaacaacgaaagctcatgcggttcctaatggtactatcgtcaaaacaatcaccaacgaccagattgaggtaactaacgctaccgagctggtacaatcctcctctacgggcgaaatctgcgacagcccgcatcagattctggatggcaaaaactgcaccctgattgatgcgctgctgggcgatccgcagtgcgatggctttcagaacaaaaaatgggatctgtttgtggaacgcagcaaagcgtatagcaactgctatccgtatgatgtgccggattatgcgagcctgcgcagcctggtggcgagcagcggcaccctggaatttaacaacgaaagctttaactggaccggcgtgacccagaacggcaccagcagcgcgtgcattcgccgcagcaaaaacagcttttttagccgcctgaactggctgacccatctgaactttaaatatccggcgctgaacgtgaccatgccgaacaacgaacagtttgataaactgtatatttggggcgtgcatcatccgggcaccgataaagatcagatttttctgtatgcgcaggcgagcggccgcattaccgtgagcaccaaacgcagccagcagaccgtgagcccgaacattggcagccgcccgcgcgtgcgcaacattccgagccgcattagcatttattggaccattgtgaaaccgggcgatattctgctgattaacagcaccggcaacctgattgcgccgcgcggctattttaaaattcgcagcggcaaaagcagcattatgcgcagcgatgcaccgatcggcaagtgcaactcagaatgtattaccccgaatggttctatacctaacgacaaacctttccaaaacgtgaaccgcattacctacggtgcctgtcccaggtatgtcaagcaaaatacactgaaactcgctacgggcatgagaaatgtggcagttgtttcagcagatgctaaaaatgccttaattgctggcggcgttgacgctaccgatgctaatggcgctgagttggtcaaaatgtcttataccgataaaaatggtaagacaattgaaggcggttatgcgcttaaagctggcgataagtattacgccgcagattacgatgaagcgacaggagcaattaaagctaaaaccacaagttatactgctgctgacggcactaccaaaacagcggctaaccaactgggtggcgtagacggtaaaaccgaagtcgttactatcgacggtaaaacctacaatgccagcaaagccgctggtcatgatttcaaagcacaaccagagctggcggaagcagccgctaaaaccaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattttgcagcaggccggtacttccgttctggcgcaggctaaccaggtcccgcagaacgtgctgagcctgttacgttagctcgag

SEQ ID NO: 32 - protein, 711 aa, Artificial Sequence
  HL402 STF2R3.HA1s Perth (Short version of HL399-N-17aa; C-5aa)
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAHAVPNGTIVKTITNDQIEVTNATELVQSSSTGEICDSPHQILDGKNCTLIDALLGDPQCDGFQNKKWDLFVERSKAYSNCYPYDVPDYASLRSLVASSGTLEFNNESFNWTGVTQNGTSSACIRRSKNSFFSRLNWLTHLNFKYPALNVTMPNNEQFDKLYIWGVHHPGTDKDQIFLYAQASGRITVSTKRSQQTVSPNIGSRPRVRNIPSRISIYWTIVKPGDILLINSTGNLIAPRGYFKIRSGKSSIMRSDAPIGKCNSECITPNGSIPNDKPFQNVNRITYGACPRYVKQNTLKLATGMRNVAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLR

SEQ ID NO: 33 - protein, 729 aa, Artificial Sequence
  STF2.HA1-2 PR8
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAYANNGTTLDVSGLDDAAIKAATGGTNGTASVTGGAVKFDADNNKYFVTIGGFTGADAAKNGDYEVNVATGDTVTLAAGATKTTMPAGATTKTEVQELKDTPAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLAKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITS

SEQ ID NO: 34 - protein, 564 aa, Artificial Sequence
  A/PUERTO RICO/8/34 HA
MKANLLVLLSALAAADADTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNIPSIQSRGLFGAIAGFIEGGWTGMIDGWYGYHHQNEQGSGYAADQKSTQNAINGITNKVTVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGVKLESMGIYQILAIYSTVASSLVLLVSLGAISFWMCSNGSLQCRICI

SEQ ID NO: 35 - protein, 518 aa, Artificial Sequence
  STF2.6HIS
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAYANNGTTLDVSGLDDAAIKAATGGTNGTASVTGGAVKFDADNNKYFVTIGGFTGADAAKNGDYEVNVATGDTVTLAAGATKTTMPAGATTKTEVQELKDTPAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLRLVPRGSHHHHHH

SEQ ID NO: 36 - protein, 405 aa, Artificial Sequence
  STF2R3
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLR

SEQ ID NO: 37 - protein, 405 aa, Artificial Sequence
  STF2R3.2X
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLA

SEQ ID NO: 38 - protein, 518 aa, Artificial Sequence
  STF2
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAYANNGTTLDVSGLDDAAIKAATGGTNGTASVTGGAVKFDADNNKYFVTIGGFTGADAAKNGDYEVNVATGDTVTLAAGATKTTMPAGATTKTEVQELKDTPAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKADAALAQVDALRSDLGAVQNRFNSAITNLGNTVNNLSEARSRIEDSDYATEVSNMSRAQILQQAGTSVLAQANQVPQNVLSLLRLVPRGSHHHHHH

SEQ ID NO: 39 - protein, 729 aa, Artificial Sequence
  STF2.HA1-2 VN
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAYANNGTTLDVSGLDDAAIKAATGGTNGTASVTGGAVKFDADNNKYFVTIGGFTGADAAKNGDYEVNVATGDTVTLAAGATKTTMPAGATTKTEVQELKDTPAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLAGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSE

SEQ ID NO: 40 - protein, 629 aa, Artificial Sequence
  STF2R3.HA1-2 VN
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSESAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLR

SEQ ID NO: 41 - protein, 730 aa, Artificial Sequence
  STF2.HA1-2 CA07
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAYANNGTTLDVSGLDDAAIKAATGGTNGTASVTGGAVKFDADNNKYFVTIGGFTGADAAKNGDYEVNVATGDTVTLAAGATKTTMPAGATTKTEVQELKDTPAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLASGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRDQEGRMNYYWTLVEPGDKITFEATGNLVVPRYAFAMERNAGSGIIIS

SEQ ID NO: 42 - protein, 627 aa, Artificial Sequence
  STF2R3.HA1-2 CA07
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRDQEGRMNYYWTLVEPGDKITFEATGNLVVPRYAFAMERNAGSGIIISVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLR

SEQ ID NO: 43 - protein, 851 aa, Artificial Sequence
  STF2R3.2XHA1-2 CA07
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRDQEGRMNYYWTLVEPGDKITFEATGNLVVPRYAFAMERNAGSGIIISVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLRSGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRDQEGRMNYYWTLVEPGDKITFEATGNLVVPRYAFAMERNAGSGIIIS

SEQ ID NO: 44 - protein, 729 aa, Artificial Sequence
  STF2.HA1-2 SI
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAYANNGTTLDVSGLDDAAIKAATGGTNGTASVTGGAVKFDADNNKYFVTIGGFTGADAAKNGDYEVNVATGDTVTLAAGATKTTMPAGATTKTEVQELKDTPAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLAKGIAPLQLGNCSVAGWILGNPECELLISRESWSYIVEKPNPENGTCYPGHFADYEELREQLSSVSSFERFEIFPKESSWPNHTTTGVSASCSHNGESSFYKNLLWLTGKNGLYPNLSKSYANNKEKEVLVLWGVHHPPNIGDQRALYHKENAYVSVVSSHYSRKFTPEIAKRPKVRDQEGRINYYWTLLEPGDTIIFEANGNLIAPRYAFALSRGFGSGIINS